(19) United States
(12) Patent Application Publication
(10) Pub. No.: US 2009/0163851 A1
(43) Pub. Date: Jun. 25, 2009

(54) OCCLUSIVE MATERIAL REMOVAL DEVICE HAVING SELECTIVELY VARIABLE STIFFNESS

(76) Inventors: Kenneth A. Holloway, Atlanta, GA (US); Aadel Al-Jadda, Atlanta, GA (US); Sanjay Singh Yadav, Sandy Springs, GA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/272,069

(22) Filed: Nov. 17, 2008

Related U.S. Application Data
(60) Provisional application No. 61/014,779, filed on Dec. 19, 2007.

(51) Int. Cl. A61B 17/221; A61M 29/02

(52) U.S. Cl. 604/22; 606/200

(57) ABSTRACT

Apparatus and methods for removal of obstructing or occluding material, such as tissue, from within bodily lumens via a minimally invasive approach are disclosed. In one embodiment, an apparatus, such as a medical device, includes an elongate member and a tissue disrupter. The elongate member is configured to be at least partially disposed within a bodily lumen. The tissue disrupter is coupled to a distal end portion of the elongate member. The tissue disrupter is configured to be selectively stiffened and is configured to dislodge a tissue from within the bodily lumen.

### CROSS-REFERENCE TO RELATED APPLICATIONS

This application claims priority to U.S. Provisional Application Ser. No. 61/014,779, entitled “Thrombus Removal Device Having Hydraulic Fiber Mesh,” filed Dec. 19, 2007, which is incorporated herein by reference in its entirety.

### BACKGROUND

The present invention is related generally to medical devices and methods, and particularly to the removal of obstructing or occlusive material from bodily lumens via a minimally invasive approach.

Strokes are a leading cause of death and disability in the world. There are two different types of strokes, hemorrhagic and ischemic. Hemorrhagic stroke occurs when a blood vessel in the brain ruptures, thereby releasing blood into the surrounding brain tissue causing damage. Ischemic strokes are caused by blockages of the vessels that bring blood to the brain. Ischemic strokes can be further divided into two primary types: thrombotic and embolic. Both types of ischemic strokes can eventually result in a thrombus that blocks distal blood flow. Embolic strokes are caused by clots that form in the peripheral or coronary vasculature and travel to the brain through the vascular system until they become lodged in the brain vessels. Thrombotic strokes are caused by blood clots that form in the vessels supplying blood to the brain.

Thrombotic occlusions can form when a plaque in the vessel grows over time slowly reducing blood flow through the vessel. The anatomical locations of the occlusions are often found in the internal carotid, middle cerebral, anterior cerebral, vertebral or basilar arteries. These arteries can be very tortuous (e.g., often having 180 degree turns) and delicate. The tortuousness and delicacy of the vessels can make the treatment of occlusions therein very difficult and dangerous.

Some known procedures for treating ischemic strokes include delivering a lytic agent intravenously. The effectiveness of such known treatments, however, can be limited if the lytic agent is not delivered within three hours from onset of the stroke. Moreover, such known procedures can cause bleeding in the brain, thereby causing additional damage to the brain.

Thus, a need exists for improved apparatus and methods for removing obstructing material from bodily lumens via a minimally invasive approach.

### SUMMARY

Apparatus and methods for removal of obstructing or occluding material, such as tissue, from within a bodily lumen are disclosed. In some embodiments, an apparatus includes an elongate member and a tissue disruptor. The elongate member is configured to be at least partially disposed within a bodily lumen. The tissue disrupter is coupled to a distal end portion of the elongate member. The tissue disrupter is configured to be selectively stiffened and is configured to dislodge a tissue from within the bodily lumen.

### BRIEF DESCRIPTION OF THE DRAWINGS

FIGS. 1 and 2 are schematic illustrations of an apparatus according to an embodiment in a first configuration and second configuration, respectively.

FIGS. 3 and 4 are side views of a distal portion of an apparatus according to an embodiment in a first configuration and a second configuration, respectively.

FIG. 5 is a perspective view of the portion of the apparatus of FIG. 3 labeled as region Z.

FIG. 6 is a cross-sectional view of a portion of the apparatus of FIGS. 3 and 4 taken along line X₁-X₁ in FIG. 4.

FIG. 7 is a cross-sectional view of the portion of the apparatus of FIG. 5 taken along line X₂-X₂.

FIG. 8 is a side view of a proximal portion of the apparatus of FIG. 3.

FIG. 9 is an illustration of a bodily lumen having an occlusive tissue and having a portion of an apparatus according to an embodiment disposed therein.

FIGS. 10-12 are side views of the apparatus of FIGS. 3-8 in use in the bodily lumen of FIG. 9.

FIG. 13 is a flowchart of a method according to an embodiment.

FIG. 14 is a perspective view of a portion of an apparatus according to an embodiment.

FIG. 15 is a side view of the portion of the apparatus of FIG. 14.

FIG. 16 is a side view of the portion of the apparatus of FIG. 14 including a filter.

FIG. 17 is a flowchart of a method according to an embodiment.

FIGS. 18 and 19 are perspective views of a portion of an apparatus according to an embodiment in a first configuration and a second configuration, respectively.

FIG. 20 is a flowchart of a method according to an embodiment.

FIG. 21 is a flowchart of a method according to an embodiment.

FIGS. 22 and 23 are a perspective view and an end view, respectively, of a portion of an apparatus according to an embodiment.

FIGS. 24 and 25 are a perspective view and a side view, respectively, of a distal end portion and a proximal end portion, respectively, of an apparatus according to an embodiment.

FIGS. 26A-26C are cross-sectional views of a portion of apparatus according to embodiments.

### DETAILED DESCRIPTION

Apparatus and methods for removal of obstructing or occluding material from within a bodily lumen are described herein. In some embodiments, an apparatus is configured to engage and dislodge occluding material within a bodily lumen, such as within the vasculature of a patient. For example, the apparatus can be configured to dislodge a thrombus from within a blood vessel.

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to the direction closer to and away from, respectively, a user (e.g., surgeon, physician, nurse, technician, etc.) who would insert the medical device into the patient, with the tip-end (i.e., distal end) of the device inserted inside a patient's body first. Thus, for example, the end of a medical device first inserted inside the patient's body would be the distal end, while the opposite end of the medical device (e.g., the end of the medical device being operated by the user) would be the proximal end of the medical device.

As used herein, the term “stiffness” relates to an object's resistance to deflection, deformation, and/or displacement by an applied force. For example, a catheter with greater stiffness is more resistant to deflection, deformation and/or displacement when exposed to a force than a catheter having a lower stiffness. Similarly stated, a catheter having a higher stiffness can be characterized as being more rigid than a catheter having a lower stiffness. In some embodiments, the stiffness of an object can be characterized by the object's linear stiffness. Linear stiffness can be characterized in terms of the amount of force applied to the object and the resulting distance through which a first portion of the object deflects, deforms, and/or displaces with respect to a second portion of the object. When characterizing the linear stiffness of an object, the deflected distance may be measured as the deflection of a portion of the object different than the portion of the object to which the force is directly applied. Said another way, in some objects, the point of deflection is distinct from the point where force is applied.

In some embodiments, the stiffness of an object can be characterized by the object's rotational (or torsional) stiffness. Rotational stiffness can be characterized in terms of the torque (or “moment”) applied to the object and the resulting rotation of a first portion of the object with respect to a second portion of the object. For example, the moment can be measured in Newton-meters or pound-inches. The rotation of the object is a unit of angle. For example, the rotation can be measured in radians or degrees. Thus, in some embodiments, the rotational stiffness of an object can be measured in units of Newton-meters per radian or pound-inches per degree.

Stiffness is an extensive property of the object being described, and thus is dependent upon the material from which the object is formed and certain physical characteristics of the object (e.g., shape and boundary conditions). For example, the stiffness of an object can be increased or decreased by selectively including in the object a material having a desired modulus of elasticity. The modulus of elasticity is an intensive property of the constituent material and describes an object's tendency to elastically (i.e., non-permanently) deform in response to an applied force. A material having a high modulus of elasticity will not deflect as much as a material having a low modulus of elasticity in the presence of an equally applied force. Thus, the stiffness of the object can be increased, for example, by introducing into the object and/or constructing the object of a material having a high modulus of elasticity. In another example, the stiffness of the object can be increased or decreased by changing the flexural modulus of a material of which the object is constructed. Flexural modulus is used to describe the ratio of the applied stress on an object in flexure to the corresponding strain in the outermost portions of the object. The flexural modulus, rather than the modulus of elasticity, is used to characterize certain materials, for example plastics, that do not have material properties that are substantially linear over a range of conditions. An object with a first flexural modulus is less elastic and has a greater strain on the outermost portions of the object than an object with a second flexural modulus lower than the first flexural modulus. Thus, the stiffness of an object can be increased by including in the object a material having a high flexural modulus.

The stiffness of an object can also be increased or decreased by changing a physical characteristic of the object, such as the shape or cross-sectional area of the object. For example, an object having a length and a cross-sectional area may have a greater stiffness than an object having an identical length but a smaller cross-sectional area. Thus, the stiffness of the object can be increased by increasing and/or changing the shape of the cross-sectional area of the object.

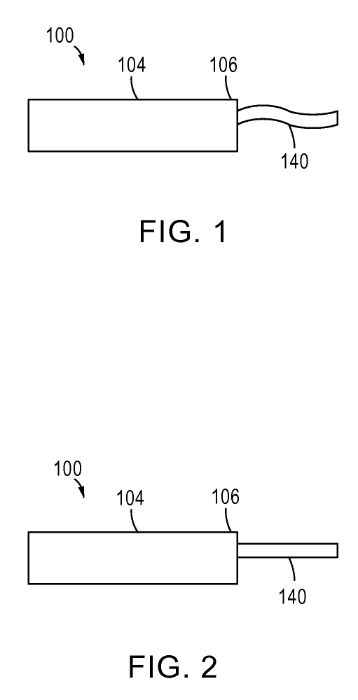

FIGS. 1 and 2 are schematic illustrations of an apparatus 100 according to an embodiment in a first configuration and a second configuration, respectively. The apparatus 100 includes an elongate member 104 and a tissue disruptor 140. The elongate member 104, which can be, for example, a catheter, is configured to be at least partially disposed within a bodily lumen (not shown in FIGS. 1 and 2). The elongate member 104 has a distal end portion 106.

The tissue disrupter 140 is coupled to the distal end portion 106 of the elongate member 104. The tissue disrupter 140 is configured to dislodge a tissue from within the bodily lumen and is configured to be selectively stiffened. Because the tissue disruptor 140 is selectively stiffenable, a user can increase and/or decrease the stiffness of the tissue disrupter as desired to insert the distal end portion into the lumen and/or dislodge the tissue, (e.g., a blood clot).

The tissue disrupter 140 has a first stiffness when the apparatus 100 is in its first configuration, as illustrated in FIG. 1, and a second stiffness different than the first stiffness when the apparatus is in its second configuration, as illustrated in FIG. 2. In some embodiments, the second stiffness is greater than the first stiffness. In this manner, the tissue disrupter 140 can be selectively stiffened after being disposed within a bodily lumen. By stiffening the tissue disruptor 140, the tissue disruptor 140 can be used to dislodge occlusive material from within the bodily lumen, as described in more detail below. A user can increase and/or decrease the stiffness of the tissue disruptor 140 until a desired stiffness is achieved.

Although the tissue disruptor 140 is illustrated in FIGS. 1 and 2 as having a first shape when the tissue disruptor is in the first configuration and a second shape different than the first shape when the tissue disruptor is in the second configuration, the shape difference is for illustrative purposes only; the tissue disruptor need not change shapes when moved between its first configuration and its second configuration. Similarly, the tissue disruptor 140 need not change in size (e.g., length, width, and/or diameter) when moved between its first configuration and its second configuration.

The tissue disruptor 140 can be selectively stiffened by any suitable mechanism. For example, in some embodiments, the tissue disruptor 140 can be selectively stiffened magnetically. For example, in some embodiments, the tissue disruptor 140 can be configured to have a first stiffness in the presence of a magnetic field and a second stiffness different than the first stiffness when the magnetic field is removed. In other embodiments, the tissue disruptor 140 is configured to have a first shape that can be moved to a second shape resulting in a greater stiffness of the tissue disruptor 140 when a magnet is brought into proximity with the apparatus 100. In still another example, the tissue disruptor 140 can be constructed of a material having a first material property associated with a first stiffness in the absence of a magnetic field and a second material property associated with a second stiffness when in the presence of a magnetic field, the second stiffness greater than the first stiffness.

In some embodiments, the tissue disruptor 140 is configured to be selectively stiffened electro-mechanically. For example, in some embodiments, the tissue disruptor 140 is configured to have a second stiffness different than a first stiffness when the tissue disruptor is exposed to an electrical current.

In some embodiments, the tissue disruptor 140 is configured to be selectively stiffened by the introduction and/or addition of a stiffening material, for example a slurry of a hardening or magnetized material. In some embodiments, the tissue disruptor 140 is selectively stiffened by changing the phase of a material forming a portion of the tissue disruptor. For example, in some embodiments, the tissue disruptor 140 can include a paraffin and can have a first stiffness when the paraffin wax is in a solid form. The tissue disruptor 140 can have a second stiffness different than the first stiffness when the paraffin is changed to a liquid.

In some embodiments, as described in more detail herein, the tissue disruptor 140 is configured to be pneumatically and/or hydraulically stiffened. For example the tissue disruptor 140 can be pneumatically stiffened by selectively conveying a gas from a source outside the patient's body to the tissue disrupter 140. In another example, the tissue disrupter 140 can be hydraulically stiffened by selectively conveying a saline solution from a source of fluid to the tissue disrupter.

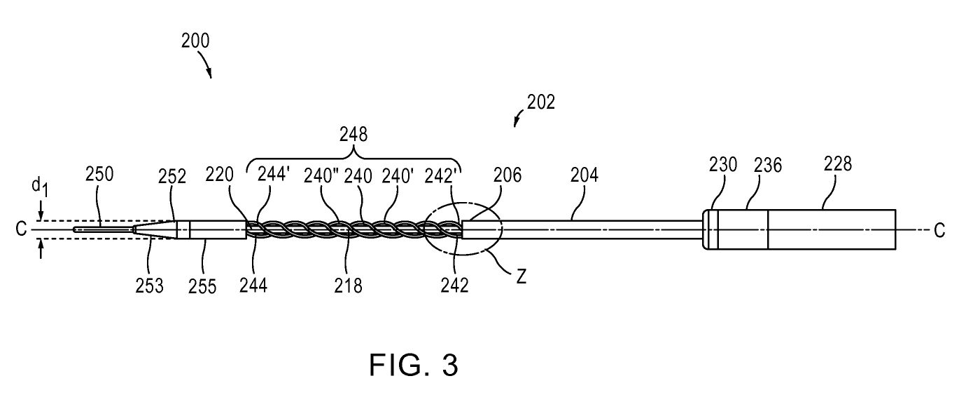

An apparatus 200 according to an embodiment is illustrated in FIGS. 3-8. At least a portion of the apparatus 200 is configured to be disposed within a bodily lumen. The apparatus 200 is configured to engage, dislodge, and/or remove occluding material from within the bodily lumen. For example, the apparatus 200 can be configured to engage, dislodge, and/or remove a thrombus from within a blood vessel.

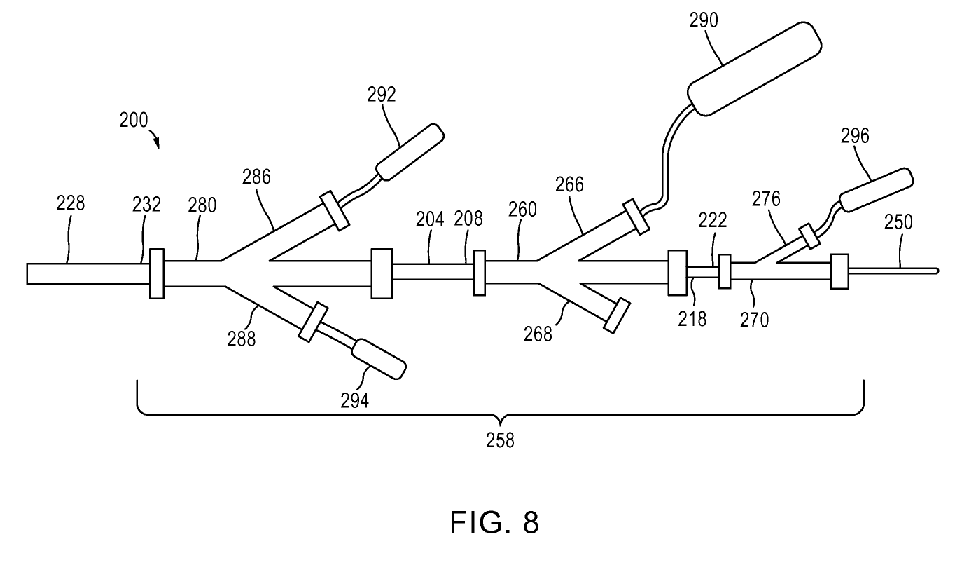

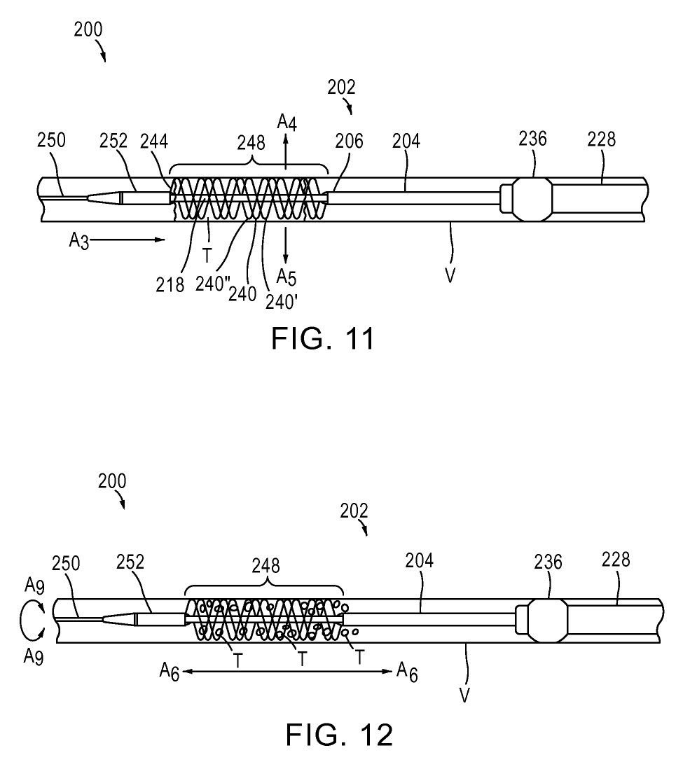

The apparatus 200 includes an elongate assembly 202, three tissue disruptors 240, 240′, 240″, and a valve assembly 258 (FIG. 8). The elongate assembly 202 is configured to be at least partially disposed within the bodily lumen L (see, e.g., FIG. 11). The elongate assembly 202 includes a first shaft 204, a second shaft 218, and an aspiration shaft 228. Each shaft, which can be, for example, a catheter, is also referred to herein as an “elongate member.”

The first shaft 204 has a distal end portion 206 and proximal end portion 208 (see, e.g., FIG. 8). The distal end portion 206 of the first shaft 204 is coupled to the tissue disruptors 240, 240′, 240″. The proximal end portion 208 of the first shaft 204 is coupled to a source of pressurized fluid 290 by a valve 260, as illustrated in FIG. 8. In some embodiments, for example, the valve 260 can be a rotating hemostatic valve, also known as a Touhy valve. The valve 260 includes a first port 266 and a second port 268. The first port 266 is coupled to the source of pressurized fluid 290. In some embodiments, the second port 268 can be coupled to a device for introducing a therapeutic agent into the body. For example, the second port 268 can be coupled to a hypodermic needle, an infusion device, or the like. In some embodiments, the second port 260 can be coupled to a device for receiving at least a portion of a fluid from the first shaft 204. For example, the second port 260 can be coupled to a pump, a hand held syringe, a vacuum, another known device for providing a suction, a receptacle for receiving a fluid, or the like. Each of the first port 266 and the second port 268 of the valve 260 can be configured to be selectively opened and/or closed by a user.

The first shaft 204 defines a central lumen 210 and three pressure lumens 212, 212′, 212″, as illustrated in FIG. 6. The central lumen 210 is configured to receive at least a portion of the second shaft 218, as described in more detail below. The pressure lumens 212, 212′, 212″ are configured to receive and/or contain a fluid from the source of pressurized fluid. Said another way, the pressure lumens 212, 212′, 212″ of the first shaft 204 can be placed in fluid communication with the source of pressurized fluid 290 via the valve 260. The fluid from the source of pressurized fluid 290 can be any fluid or fluid-like material, such as any known liquid, gas, or solid material, or combination thereof, suitable for use in a medical device within a body of a patient. For example, in some embodiments, the fluid can be saline or a saline solution, a contrast (e.g., an angiographic contrast), a radiopaque liquid mixture, or the like, or any combination thereof. In another example, in some embodiments, the fluid can be air, nitrogen, or the like, or any combination thereof. In still another example, in some embodiments, the fluid can be a solid-liquid slurry or a gel.

In use, the first shaft 204 can be selectively stiffened by a user. For example, in some embodiments, the first shaft 204 can be selectively stiffened by conveying a fluid from the source of pressurized fluid 290 into the pressure lumens 212, 212′, 212″. In other embodiments, the first shaft 204 can be selectively stiffened by changing a pressure of a fluid within the pressure lumens 212, 212′, 212″ from a first pressure to a second pressure greater than the first pressure. For example, a user can increase a pressure of the fluid contained within the pressure lumens 212, 212′, 212″ of the first shaft 204 by placing the pressure lumens 212, 212′, 212″ in fluid communication with the source of pressurized fluid 290.

In some embodiments, the portion of the first shaft 204 defining the pressure lumens 212, 212′, 212″ can be substantially non-compliant. The compliance of a material and/or an object refers to the degree to which the material and/or object can expand and/or deform beyond its nominal size. Thus, a highly compliant material can significantly elastically deform when exposed to a pressure, and a low or non-compliant material resists significant deformation when exposed to a pressure. For example, the compliance of the first shaft 204 can be characterized by the change in volume of the pressure lumens 212, 212′, 212″ as a function of the pressure within the lumens. In some embodiments, for example, the volume of the pressure lumen 212 of the first shaft 204 that is characterized as being low or non-compliant can change by zero to five percent when exposed to an internal pressure of 450 p.s.i. or less. In other embodiments, for example, the volume of the pressure lumen 212 of the first shaft 204 that is characterized as being low or non-compliant can change by zero to thirty percent when exposed to an internal pressure of 450 p.s.i. or less. In contrast, the volume of the pressure lumen 212 of the first shaft 204 that is characterized as being highly compliant can change by at least two hundred percent when exposed to an internal pressure of 450 p.s.i. or less. In other embodiments, the first shaft 204 need not be low or non-compliant.

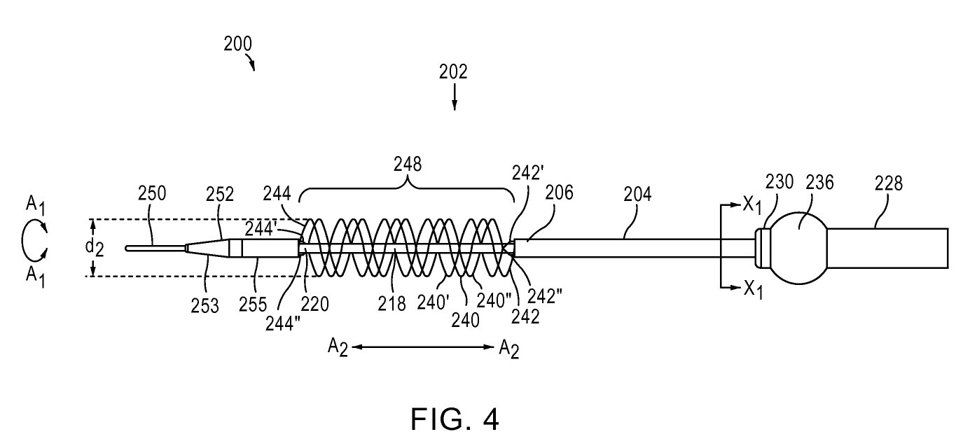

The second shaft 218 of the elongate assembly 202 has a distal end portion 220 and a proximal end portion 222 (see, e.g., FIG. 8) and defines a lumen 224. At least a portion of the second shaft 218 is movably disposed within the central lumen 210 of the first shaft 204. For example, in some embodiments, the second shaft 218 can be rotated with respect to the center line C within the central lumen 210 of the first shaft 204, as indicated by arrow A₁ in FIG. 4. In another example, in some embodiments, the second shaft 218 can be translated along the center line C, as indicated by arrow A₂ in FIG. 4. For example, the second shaft 218 can be translationally moved in a proximal and/or distal direction within the central lumen 210 of and with respect to the first shaft 204.

The distal end portion 220 of the second shaft 218 is coupled to a dilator 252. The dilator 252 has a tapered portion 253 and a coupling portion 255. The tapered portion 253 facilitates insertion of the elongate assembly 202 into the body and/or advancement of the elongate assembly 202 within the bodily lumen. The tapered portion 253 can dilate, displace and/or stretch tissue without cutting the tissue. In other embodiments, the dilator can be configured to pierce and/or cut tissue. The coupling portion 255 of the dilator 252 is also configured to couple the tissue disruptors 240, 240′, 240″ to the second shaft 218, as described in more detail below.

The lumen 224 of the second shaft 218 is configured to receive a guidewire 250 and/or to allow passage of an irrigation fluid therethrough. As illustrated in FIG. 8, the proximal end portion 222 of the second shaft 218 is coupled to a valve 270, such as a Touhy valve similar to the valve 260 as described above. The valve 270 includes a port 276 configured to be connected to an irrigation source 296. The irrigation source 296 can provide an irrigation fluid (e.g., a liquid or gas) to irrigate and/or cleanse a treatment area within the bodily lumen. For example, the irrigation fluid can be introduced into the apparatus 200 via port 276 and can pass through the lumen 224 of the second shaft 218 to the treatment site to help irrigate and/or wash an area within the bodily lumen at which the tissue is dislodged.

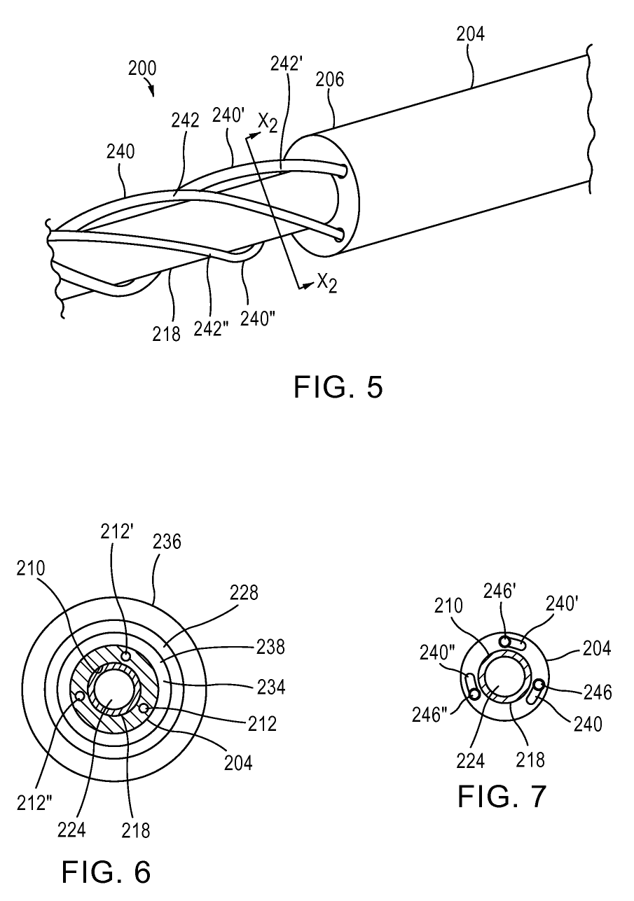

The tissue disruptors 240, 240′, 240″ of the apparatus 200 form a set of tissue disruptors 248 configured to dislodge the tissue from within the bodily lumen. For example, as described in more detail herein, the set of tissue disruptors 248 can be used to dislodge a thrombus from within a blood vessel. Each tissue disrupter 240, 240′, 240″ has a proximal end portion 242, 242′, 242″ (best shown in FIGS. 4 and 5) and a distal end portion 244, 244′, 244″ (best shown in FIG. 4) and defines a lumen 246, 246′, 246″ (best shown in FIG. 7).

As illustrated in FIGS. 3-5, the proximal end portion 242, 242′, 242″ of each tissue disrupter 240, 240′, 240″ is coupled to the distal end portion 206 of the first shaft 204 such that the lumen 246, 246′, 246″ of each tissue disrupter is in fluid communication with a respective pressure lumen 212, 212′, 212″ of the first shaft. In this manner, fluid can be conveyed from the source of pressurized fluid 290 to the lumen 246, 246′, 246″ of each tissue disrupter 240, 240′, 240″ via the pressure lumens 212, 212′, 212″ of the first shaft 204. The proximal end portion 242, 242′, 242″ of each tissue disrupter 240, 240′, 240″ can be coupled to the first shaft 204 by any suitable coupling mechanism. For example, the proximal end portion 242, 242′, 242″ of the tissue disrupter 240, 240′, 240″ can be coupled to the first shaft by an adhesive, laser weld, a mechanical fastener, or the like, or any combination thereof.

The distal end portion 244, 244′, 244″ of each tissue disrupter 240, 240′, 240″ is configured to prevent escape of the fluid within the lumen 246, 246′, 246″ of the tissue disrupter 240, 240′, 240″. Similarly stated, the distal end portion 244, 244′, 244″ of each tissue disrupter 240, 240′, 240″ is fluidically isolated from a region outside of the tissue disrupter 240, 240′, 240″. For example, the lumen 246, 246′, 246″ at the distal end portion 244, 244′, 244″ of the tissue disruptors 240, 240′, 240″ can be sealed closed. In another example, the tissue disruptors 240, 240′, 240″ can be configured to define a lumen extending only partially therethrough. The closed distal end portion 244, 244′, 244″ allows the user to selectively control the pressure of the fluid within the lumen 246, 246′, 246″ by conveying and/or releasing a portion of the fluid from the lumens 246, 246′, 246″ at the proximal end portions 242, 242′, 242″ of the tissue disruptors 240, 240′, 240″. In some embodiments, the pressure of the fluid within the lumens 246, 246′, 246″ can be adjusted without conveying a portion of the fluid into and/or releasing a portion of the fluid from the lumens 246, 246′, 246″. Similarly stated, in some embodiments, the tissue disruptors 240, 240′, 240″ define a closed system (e.g., a system that is fluidically isolated from an area outside of the tissue disruptors 240, 240′, 240″).

The distal end portion 244, 244′, 244″ of each tissue disruptor 240, 240′, 240″ is coupled to the distal end portion 220 of the second shaft 218 by the coupling portion 255 of the dilator 252, as illustrated in FIG. 3. This arrangement allows the user to move the distal end portion 244, 244′, 244″ of each tissue disrupter 240, 240′, 240″ with respect to the first shaft 204 and/or the proximal end portion 242, 242′, 242″ of each tissue disruptor 240, 240′, 240″ by moving the second shaft 218 within the first shaft 204. For example, the distal end portion 244, 244′, 244″ of each tissue disrupter 240, 240′, 240″ can be translated, for example in a proximal direction, with respect to the first shaft 204 by translating the second shaft 218 with respect to the first shaft 204 in the proximal direction. In another example, the distal end portion 244, 244′, 244″ of each tissue disrupter 240, 240′, 240″ can be rotated with respect to the first shaft 204 by rotating the second shaft 218 with respect to the first shaft 204. Having two points of coupling (i.e., at the tissue disruptors' respective distal and proximal end portions 244, 244′, 244″ and 242, 242′, 242″) to the elongate assembly 202 also reduces the likelihood that a tissue disrupter 240, 240′, 240″ will be inadvertently left within the bodily lumen.

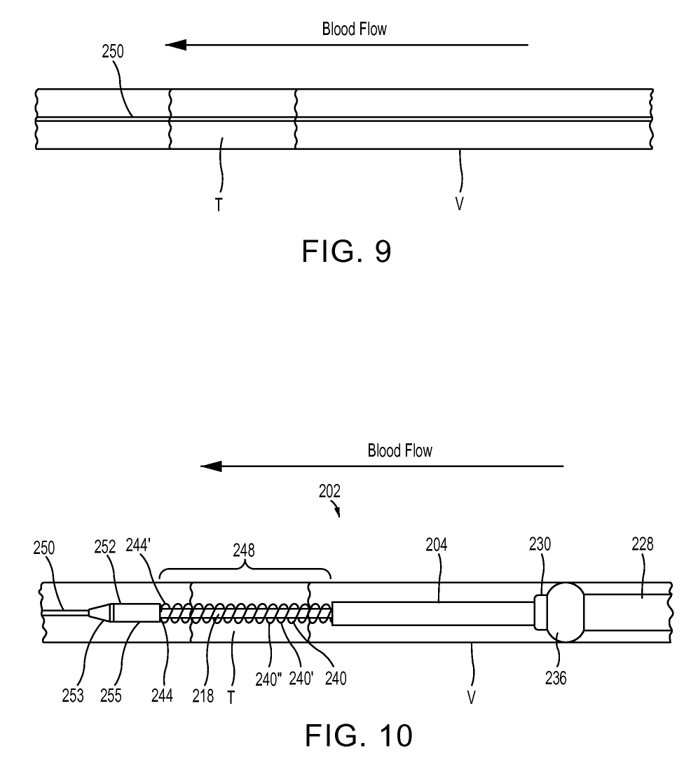

The set of tissue disruptors 248 is disposed about a portion of an exterior surface of the second shaft 218. More particularly, the set of tissue disruptors 248 is helically wound or wrapped about the distal end portion 220 of the second shaft 218. The set of tissue disruptors 248 have a first configuration in which the tissue disruptors 240, 240′, 240″ are each in contact with the exterior surface of the second shaft 218. The set of tissue disruptors 248 have a second configuration in which a portion of each tissue disruptor 240, 240′, 240″ is spaced apart from the exterior surface of the second shaft 218. Thus, the set of tissue disruptors 248 collectively has a first lateral dimension d₁ when the set of tissue disruptors is in the first configuration (as illustrated in FIG. 3). The set of tissue disruptors 248 collectively has a second lateral dimension d₂ greater than the first lateral dimension d₁ when the set of tissue disruptors is in the second configuration (as illustrated in FIG. 4). In this manner, the set of tissue disruptors 248 can be moved to its first configuration to facilitate insertion and advancement of the set of tissue disruptors 248 within the bodily lumen and can be moved to its second configuration to engage and/or dislodge occluding tissue within the bodily lumen of the patient. Movement of the set of tissue disruptors 248 from its first configuration to its second configuration is described in more detail below with reference to FIGS. 9-12.

The set of tissue disruptors 248 is configured to be selectively stiffened by a user. For example, in some embodiments, the set of tissue disruptors 248 is selectively stiffened to facilitate introduction into and advancement within the bodily lumen. The tissue disruptors 240, 240′, 240″ can be selectively stiffened by increasing and/or tightening the windings of the tissue disruptors 240, 240′, 240″ disposed about the second shaft 218. The windings are increased and/or tightened by moving the second shaft 218 with respect to the first shaft 204. For example, the second shaft 218 can be rotated with respect to the first shaft 204, as described above. As the second shaft 218 is rotated as indicated by arrow A1, the distal end portion 244, 244′, 244″ of each tissue disruptor 240, 240′, 240″ rotates relative to the proximal end portions 242, 242′, 242″, thus winding the set of tissue disruptors 248 about the second shaft. Such stiffening facilitates insertion and advancement of the set of tissue disruptors 248 into the bodily lumen. For example, use of the apparatus 200 in a procedure to remove a thrombus may first require a push force for introduction of the tissue disruptor 240 into a less tortuous blood vessel than the tortuous and delicate vessels supplying blood to the brain. The tissue disruptor 240 can be selectively stiffened within the body of the patient to facilitate advancement through this less tortuous vasculature, for example, until the tissue disrupter approaches more tortuous vasculature. Although the stiffening is described herein as rotating the second shaft 218 with respect to the first shaft 204, the stiffening can also be achieved in another manner, e.g., by rotating the first shaft 204 with respect to the second shaft 218 or simultaneously rotating each of the first shaft 204 and the second shaft 218 in opposite rotational directions.

In another example, in some embodiments, the set of tissue disruptors 248 is selectively stiffened by changing or increasing the pressure of a fluid contained within the lumen 246, 246′, 246″ of each tissue disrupter. The lumen 246, 246′, 246″ of each tissue disrupter 240, 240′, 240″ has a substantially constant volume, which allows a user to control the stiffness of the tissue disruptors by controlling a pressure of the fluid contained within the lumen of each tissue disruptor. The volume within the lumen 246, 246′, 246″ of each tissue disrupter 240, 240′, 240″ remains substantially constant despite a change in pressure of a fluid therein because the portion of each tissue disruptor defining its respective lumen is low or non-compliant. As the pressure of the fluid within the low or non-compliant tissue disrupter 240, 240′, 240″ is increased, the tissue disruptors' resistance to deflection or displacement by an applied force correspondingly increases; thus, the tissue disruptor is stiffened. Said another way, the tissue disruptors 240, 240′, 240″ have a first stiffness when the fluid within the lumen 246, 246′, 246″ of each tissue disrupter has a first pressure, and a second stiffness different than the first stiffness when the fluid within the lumen of each tissue disrupter has a second pressure different from the first pressure. As such, the set of tissue disruptors 248 is selectively stiffenable by selectively increasing the pressure of the fluid within the lumen 246, 246′, 246″ of each tissue disrupter 240, 240′, 240″.

Stiffening the tissue disruptors 240, 240′, 240″ by increasing the pressure within their respective lumens 246, 246′, 246″ can facilitate advancement of the set of tissue disruptors 248 and/or the elongate assembly 202 within the bodily lumen, similar to rotationally stiffening the tissue disruptors as described above. The stiffness of the tissue disruptors 240, 240′, 240″ can be increased, for example, when greater push force is needed to advance the apparatus 200 within the bodily lumen. As the tissue disruptors 240, 240′, 240″ approach more tortuous and/or delicate anatomy, the stiffness of the tissue disruptors can be reduced, for example by reducing the pressure of the fluid within the lumen 246, 246′, 246″ of each tissue disrupter. Stiffening the tissue disruptors 240, 240′, 240″ by increasing the pressure within their respective lumens 246, 246′, 246″ also facilitates dislodging the occlusive tissue from within the bodily lumen, as described below.

The aspiration shaft 228 of the elongate assembly 202 is configured to receive a portion of dislodged tissue and to facilitate removal of the dislodged tissue from the bodily lumen, as described in more detail below. The aspiration shaft 228 includes a distal end portion 230 (see, e.g., FIGS. 3 and 4) and a proximal end portion 232 (see, e.g., FIG. 8) and defines a lumen 234 (see, e.g., FIG. 6). At least a portion of the first shaft 204 is movably disposed within the lumen 234 of the aspiration shaft 228. For example, the first shaft 204 can be rotationally and/or translationally moved with respect to the aspiration shaft 228, similar to the movement of the second shaft 218 with respect to the first shaft 204 described above.

An inner surface of the aspiration shaft 228 and an outer surface of the first shaft 204 define a passageway 238 configured to receive a portion of the tissue when a suction is applied to the passageway. As shown in FIG. 8, an aspirator 292 is coupled to the proximal end portion 232 of the aspiration shaft 228 by a valve 280. The valve 280 can be a rotating hemostatic valve similar to the Touhy valve 260, described above. The valve 280 has a first port 286 and a second port 288. The first port 286 of the valve 280 is in fluid communication with the lumen 234 of the aspiration shaft 228, and therefore the passageway 238 defined by the aspiration shaft and the first shaft 204. The first port 286 of the valve 280 is coupled to the aspirator 292. The aspirator 292 provides a suction within the passageway 238 sufficient to withdraw a portion of the dislodged tissue from within the bodily lumen. The aspirator 292 can be a pump, hand held syringe, vacuum, or other known device for providing a suction.

An expandable member 236 is disposed on the distal end portion 230 of the aspiration shaft 228. The expandable member is configured to move from a collapsed configuration (see, e.g., FIG. 3) to an expanded configuration (see, e.g., FIG. 4). When the expandable member 236 is in the expanded configuration, the expandable member 236 has a size and/or volume greater than a size and/or volume of the expandable member 236 when in its collapsed configuration. In this manner, the expandable member 236 can substantially occlude the bodily lumen when the expandable member is in its expanded configuration, and thus substantially prevent flow of a bodily fluid therethrough (see, e.g., FIG. 11). In use, the expandable member 236 can be moved to its expanded configuration within a blood vessel to substantially prevent the flow of blood therethrough, such as while the occluding tissue is being dislodged. The expandable member is configured to be expanded by the introduction of an inflation fluid, such as a liquid or gas, into an interior chamber (not illustrated) of the expandable member. In various embodiments, the expandable member can be, for example, a non-compliant, low compliant, or high compliant balloon.

An inflation lumen (not shown) extends from the expandable member 236 of the aspiration shaft 228 to the valve 280 coupled to the proximal end portion 232 of the aspiration shaft 228. The second port 288 of the valve 280 is in fluid communication with the inflation lumen, and thus the expandable member 236. A source of inflation fluid 294 is coupled to the first port 286 of the valve 280. The source of inflation fluid 294 is configured to introduce the inflation fluid into the inflation lumen via the second port 288 of the valve 280 such that the inflation fluid is introduced into the expandable member 236 to move the expandable member to its expanded configuration. The source of inflation fluid 294 is also configured to withdraw the inflation fluid from the inflation lumen and the expandable member 236 to move the expandable member from its expanded configuration to its collapsed configuration. The source of inflation fluid 294 can be, for example, a hand held syringe.

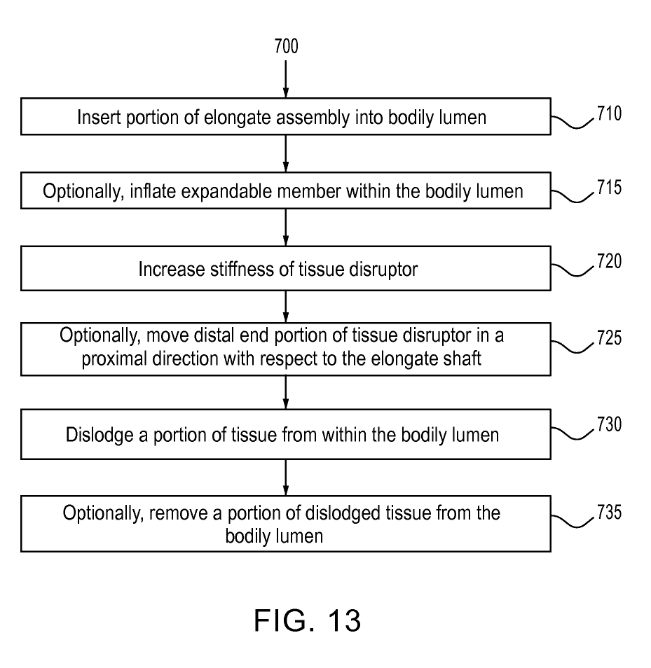

FIG. 13 is a flow chart of a method 700 of using the apparatus 200 according to an embodiment. The method illustrated in FIG. 13 is discussed with reference to FIGS. 9-12. Although the method 700 is discussed with reference to the apparatus 200, the method 700 can be performed with any suitable apparatus. For example, the method 700 can be performed by any of the medical devices disclosed herein. Referring to FIG. 9, a guidewire 250 is inserted into a bodily lumen, such as a vessel V. The vessel V can be, for example, a blood vessel that supplies blood to the patient's brain. The guidewire 250 can be inserted into the vessel V in the direction of blood flow until the guidewire is in a desired position or depth of insertion into the vessel. In some embodiments, the guidewire 250 is disposed through the occlusive tissue T.

Returning to the flowchart shown in FIG. 13, the method includes inserting at least a portion of the elongate assembly into a bodily lumen, 710. As shown in FIG. 10, a portion of the elongate assembly 202 is inserted into the vessel V in the direction of the blood flow. The elongate assembly 202 is inserted into the vessel V until the elongate assembly is positioned at the desired location within the vessel (e.g., adjacent the tissue T occluding the vessel V, proximal to the tissue T, or distal to the tissue T). In some embodiments, the first shaft 204 of the elongate assembly 202 is stiffened before being inserted into the bodily lumen. The first shaft 204 can be stiffened by any manner described herein.

Returning to the flow chart shown in FIG. 13, in some embodiments, the method optionally includes expanding an expandable member within a bodily lumen, 715. Referring to FIG. 10, the expandable member 236 is shown in the expanded configuration. When the expandable member 236 is in its expanded configuration, the expandable member contacts the inner walls of the vessel V and substantially prevents flow of bodily fluid, for example blood flow, through that portion of the vessel.

Returning to the flow chart shown in FIG. 13, the stiffness of the tissue disrupter is increased, 720. The stiffness of the tissue disruptors 240, 240′, 240″ can be increased in any manner of stiffening described herein. For example, the user can increase the stiffness of the set of tissue disruptors 248 by conveying a fluid from the source of pressurized fluid 290 into the lumen 246, 246′, 246″ of each tissue disruptor 240, 240′, 240″ via the pressure lumens 212, 212′, 212″ of the first shaft 204. In another example, the user can increase the stiffness of the tissue disruptors 240, 240′, 240″ by increasing the pressure of the fluid contained within the lumen 246, 246′, 246″ of each tissue disrupter. The pressure of the fluid within the lumen 246, 246′, 246″ of the tissue disruptor 240, 240′, 240″ can be adjusted (increased and/or decreased) until the desired stiffness of the set of tissue disruptors 248 is achieved.

Returning to the flow chart shown in FIG. 13, in some embodiments, the method optionally includes moving a distal end portion of the tissue disrupter in a proximal direction with respect to an elongate shaft, 725. As shown in FIG. 11, the distal end portion 244, 244′, 244″ of each tissue disrupter 240, 240′, 240″ can be translated in a proximal direction (indicated by arrow A₃) with respect to the first shaft 204, e.g., by moving the second shaft 218 in a proximal direction with respect to the first shaft. The first shaft 204 can remain substantially stationary within this vessel V as the respective distal end portions 244, 244′, 244″ of the tissue disruptors 240, 240′, 240″ are moved in the proximal direction. Thus, the distal end portions 244, 244′, 244″ of the tissue disruptors 240, 240′, 240″ are moved proximally (with the dilator 252 and the second shaft 218) with respect to proximal end portions 242, 242′, 242″ of the tissue disruptors 240, 240′, 240″. As the distal end portion 244, 244′, 244″ of each tissue disrupter 240, 240′, 240″ moves proximally, a portion of each tissue disrupter moves in a radial direction (indicated by arrows A₄ & A₅) away from the center line (not shown in FIG. 11) of the first shaft 204 and towards the wall of the vessel V. In this manner, the set of tissue disruptors 248 is moved from its first configuration having the first lateral dimension d₁ to its second configuration having the second lateral dimension d₂, as previously described. The tissue disruptors 240, 240′, 240″ are each configured to contact a wall or interior surface of the vessel V and/or the tissue T on the interior surface or wall of the vessel V when in the second configuration. For example, in some embodiments, the tissue disruptors 240, 240′, 240″ can engage a thrombus on an interior wall of the vessel V without shaving or otherwise damaging the wall of the vessel V.

Returning to the flow chart shown in FIG. 13, a portion of the bodily tissue is dislodged from within the bodily lumen, 730. Referring to FIG. 12, the set of tissue disruptors 248 is configured to disrupt, break up, macerate, and/or dislodge a tissue T from within the bodily lumen of the patient. For example, a user can dislodge the tissue T by moving the elongate assembly 202, and thus the set of tissue disruptors 248, to dislodge the tissue T with the stiffened and/or radially expanded set of tissue disruptors 248. For example, as the tissue disruptors 240, 240′, 240″ are moved towards the wall of the vessel V, the tissue disruptors 240, 240′, 240″ engage and/or dislodge the tissue T from the vessel V. Additionally, a portion of the apparatus 200 including the set of tissue disruptors 248 can be moved in at least one of a proximal direction, a distal direction, and/or a rotational direction as the tissue disruptors 240, 240′, 240″ engage the tissue T to agitate and dislodge the tissue T. For example, the set of tissue disruptors 248 can be alternatively moved in the proximal and distal directions (indicated by arrow A₆ in FIG. 12), i.e., a reciprocating motion, until a desired portion of the tissue T has been successfully dislodged from the bodily lumen. In another example, the set of tissue disruptors 248 can be moved in a first rotational direction (i.e., clockwise) and a second rotational direction (i.e., counter-clockwise) until the tissue T has been dislodged. In still another example, the set of tissue disruptors 248 can be moved in at least the first rotational direction and in the proximal direction (i.e., a corkscrew motion) until the tissue T has been dislodged. Moreover, in the event the tissue T requires more force or friction to become dislodged from the bodily lumen, the stiffness of the tissue disruptors 240, 240′, 240″ can be further increased.

The set of tissue disruptors 248 are configured to allow a bodily fluid to pass between the set of tissue disruptors 248 when the set of tissue disruptors 248 is in its second configuration. Said another way, the set of tissue disruptors 248 does not block the bodily lumen or cut-off flow of a bodily fluid therethrough when the tissue disruptors 240, 240′, 240″ are in contact with the vessel V wall. More particularly, as discussed above, portions of the tissue disruptors 240, 240′, 240″ are spaced apart from the second shaft 218 when the set of tissue disruptors 240, 240′, 240″ is in the second configuration. Thus, a flow path for bodily fluid exists between the portions of the tissue disruptors 240, 240′, 240″ and the second shaft 218. Additionally, a flow path for bodily fluid exists between each tissue disrupter 240, 240′, 240″ of the set of tissue disruptors 248 because the helical coils of each tissue disrupter 240, 240′, 240″ are axially spaced from the other helical coils, leaving a passageway, gap, or other opening between the helically wound tissue disruptors.

The set of tissue disruptors 248 can be used to capture a portion of the dislodged tissue T. Because the set of tissue disruptors 248 defines the space between the helical coils or windings of the tissue disruptors 240, 240′, 240″, the set of tissue disruptors 248 can capture a portion of the dislodged tissue T within the spaces between the helical coils. Said another way, the set of tissue disruptors 248 can engage the bodily tissue T such that movement of the tissue T relative to the set of tissue disruptors 248 is limited. Additionally, the set of tissue disruptors 248 can capture a portion of the dislodged tissue T between the tissue disruptors 240, 240′, 240″ and the second shaft 218. For example, the distal end portion 244, 244′, 244″ of each tissue disrupter 240, 240′, 240″ can be moved distally such that portions of the tissue disruptors 240, 240′, 240″ of the set of tissue disruptors 248 moves radially towards the second shaft 218 until a portion of the dislodged tissue T is captured between the tissue disruptors 240, 240′, 240″ and the second shaft 218.

Returning to the flow chart shown in FIG. 13, in some embodiments, the method optionally includes removing a portion of the dislodged tissue T from the bodily lumen, 735. For example, as illustrated in FIG. 12 and described above, a portion of the tissue T is captured within the coils or windings of the tissue disruptors 240, 240′, 240″ and/or between the set of tissue disruptors 248 and the second shaft 218. The tissue disruptors 240, 240′, 240″ can be removed from the bodily lumen of the patient with the captured tissue T. In some embodiments, a portion of the dislodged tissue T can be aspirated from within the vessel V via the aspiration shaft 228. For example, the source of aspiration 292 (not shown in FIG. 12) is used to apply a suction to the passageway 234 defined by the aspiration shaft 228 and the first shaft 204 such that a portion of the tissue T is received in the passageway 238 and is removed from the vessel V. In some embodiments, a portion of the elongate assembly 202 is moved in a proximal direction such that the set of tissue disruptors 248 and captured tissue T are proximate to the aspiration shaft 228, for example, to facilitate aspiration of the dislodged and/or captured tissue T.

The expandable member 236 can then be moved to its collapsed configuration to permit passage of bodily fluid through the bodily lumen. For example, the expandable member 236 can be collapsed (or deflated) to allow blood flow to resume through the vessel V. If desired, the user can then use the apparatus 200 to remove tissue T at a different occlusion site within the patient's body. For example, the user can remove at least a portion of the apparatus 200 from the vessel V and then navigate the portion of the apparatus 200 through the patient's vasculature to a different occlusion site. Otherwise, the user can remove the apparatus 200 from the body of the patient.

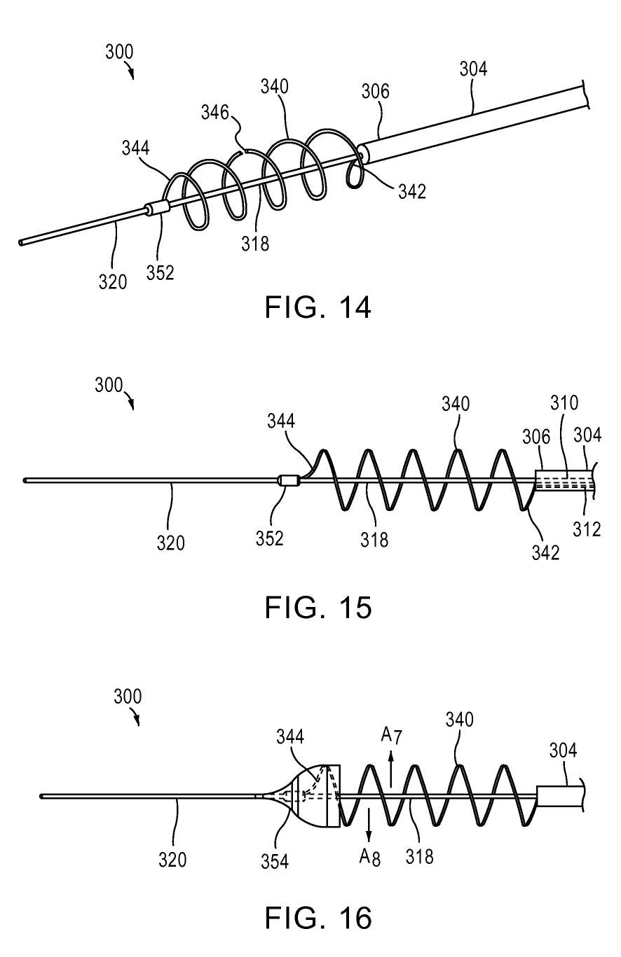

An apparatus 300 according to another embodiment is illustrated in FIGS. 14-16. The apparatus 300 includes a first elongate member 304, a second elongate member 318, a fiber 340, and a filter 354. The filter 354 is not shown in FIGS. 14 and 15 for clarity of illustration purposes. The first elongate member 304 is similar in many respects to the first shaft 204 described above with respect to FIGS. 3-8, and is therefore not described in detail. The first elongate member 304 includes a distal end portion 306 and a proximal end portion (not shown) and defines a first lumen 310 and a second lumen 312 (shown in dashed lines in FIG. 15). The first lumen 310 extends along a center line (not shown) defined by the first elongate member 304. The second lumen 312 extends from the proximal end portion to the distal end portion 306 of the first elongate member 304 and is non-coaxial with the first lumen 310. The proximal end portion of the first elongate member 304 is coupled to a source of pressurized fluid (not shown) of the types shown and described above. The distal end portion 306 of the first elongate member 304 is coupled to the fiber 340.

The fiber 340 is configured to disrupt or dislodge a tissue T within a bodily lumen. The fiber 340 is configured to be selectively stiffened, as described above with respect to tissue disruptors 240, 240′, 240″. The fiber 340 has a proximal end portion 342 and a distal end portion 344 and defines a lumen 346. The proximal end portion 342 of the fiber 340 is coupled to the distal end portion 306 of the first elongate member 304. The lumen 346 of the fiber 340 is in fluid communication with the second lumen 312 of the first elongate member 304.

The fiber 340 has a first stiffness when in a first configuration and a second stiffness different than the first stiffness when in a second configuration. The fiber 340 is configured to dislodge a tissue T within the bodily lumen when the fiber 340 is in its second configuration. At least a portion of the fiber 340 is configured to move in a direction substantially normal to the center line of the first elongate member 304 when the fiber 340 is moved from its first configuration to its second configuration, as described above with respect to the set of tissue disruptors 248, and as described in more detail below.

The second elongate member 318 has a proximal end portion (not illustrated) and a distal end portion 320. In the embodiment illustrated in FIGS. 14-16, the second elongate member 318 is a guidewire. At least a portion of the second elongate member 318 is movably disposed within the first lumen 310 of the first elongate member 304 such that the second elongate member 318 can translate and/or rotate within the first elongate member 304, as described above. The distal end portion 344 of the fiber 340 is coupled to the second elongate member 318, for example by crimping the fiber to the distal end portion 320 of the second elongate member 318 with a marker 352, as illustrated in FIG. 14. The marker 352 can be, for example, a radiopaque marker configured to help the user monitor the position or location of the apparatus 300 within a patient's body.

The second elongate member 318 can be moved axially in a proximal direction to move the portion of the fiber 340 substantially normal to the center line. As used herein, the term “substantially normal” means a direction that is substantially perpendicular, or at a 90 degree angle, to the referenced object (for example, the center line). The fiber 340 is configured to engage the tissue T when the portion of the fiber is moved substantially normal towards the wall of the bodily lumen.

The filter 354 is coupled to the second elongate member 318. The filter 354 is movable from an undeployed configuration (not shown) in which the filter has a narrow profile to a deployed configuration in which the filter has an expanded profile (as illustrated in FIG. 16). The filter 354 is disposed over a portion of the distal end portion 344 of the fiber 340 and is introduced into a bodily lumen when in the undeployed configuration. The filter 354 is then moved to its deployed configuration when it is positioned at or proximate to a desired location within the bodily lumen. The filter 354 can be deployed, for example, by moving the fiber 340 substantially normal to the center line such that a distal end portion 344 of the fiber moves the filter 354 towards its deployed configuration. In its deployed configuration, the filter 354 is configured to allow passage of a bodily fluid (e.g., blood) therethrough and to capture dislodged tissue T to prevent the dislodged tissue T from traveling downstream (distally) in the bodily lumen. For example, the filter 354 can be constructed from a permeable or semi-permeable material. The filter can be constructed of any suitable material, such as polyurethane, silicone, or plastic.

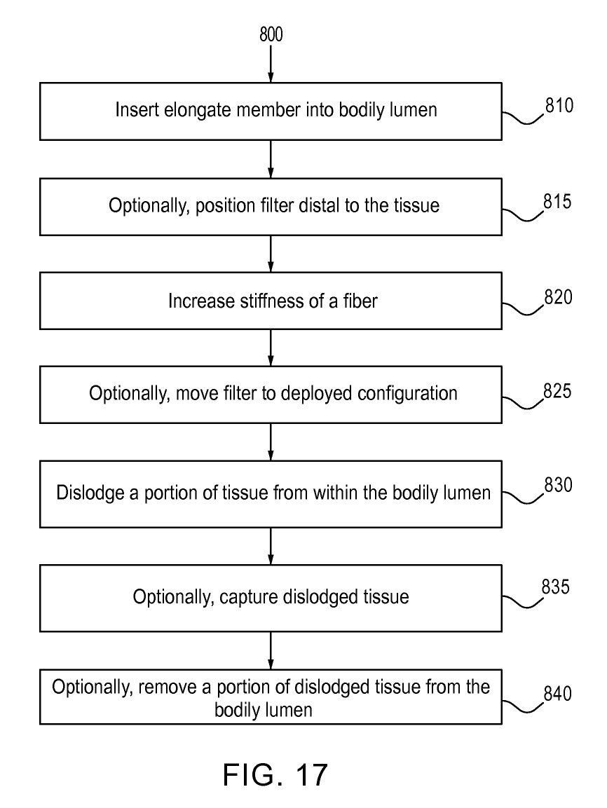

FIG. 17 is a flow chart of a method 800 of removing a tissue T within a bodily lumen according to an embodiment. The method illustrated in FIG. 17 is discussed with reference to apparatus 300 shown in FIGS. 14-16. Although the method 800 is discussed with reference to the apparatus 300, the method 800 can be performed with any suitable apparatus. For example, the method 800 can be performed by any of the medical devices disclosed herein. The method includes inserting an elongate member into a bodily lumen, 810. For example, a user can insert the second elongate member 318 of apparatus 300 into the bodily lumen. The second elongate member 318 can be a guidewire. In some procedures, such as a procedure to treat an occlusion in the internal carotid artery, the second elongate member 318 is inserted into the patient's body via the femoral artery, and the second elongate member 318 is advanced to the site of occlusive material within the internal carotid artery. The user can verify the position of the second elongate member 318 by detecting the position of the marker 352.

Returning to the flow chart shown in FIG. 17, in some embodiments, the method optionally includes positioning a filter distal to the tissue T to be dislodged and/or removed, 815. For example, the second elongate member 318 can be advanced through the occlusive tissue T such that the filter 354 of the apparatus 300 is positioned distal to the tissue T or at a distal end of the tissue T. By positioning the filter 354 distally of the tissue T, the filter 354 can prevent dislodged tissue T from moving distally in the vessel (e.g., in the direction of blood flow). When positioning the filter 354 with respect to the tissue T, the fiber 340 can be inserted through the tissue T until at least a portion of the fiber 340 is distal to the tissue T, or at least a portion of the fiber 340 can be positioned within the tissue T.

Returning to the flow chart shown in FIG. 17, the stiffness of a fiber is increased, 820. The stiffness of the fiber can be increased in any manner described herein. For example, the fiber 340 can be moved from its first configuration having its first stiffness to its second configuration having its second stiffness greater than the first stiffness, by increasing the pressure of a fluid within the lumen of the fiber.

Returning to the flow chart shown in FIG. 17, in some embodiments, the method optionally includes moving the filter to a deployed configuration, 825. For example, in some embodiments, the distal end 344 of the fiber 340 is moved proximally towards the first elongate member 304 such that a portion of the fiber moves substantially normal (as indicated by arrows A₇ & A₈ in FIG. 16) to the center line (not shown in FIGS. 14-16) of the first elongate member 304. A portion of the fiber 340 moving substantially normal to the center line moves the filter 354 to its deployed configuration. In other embodiments, the filter 354 is deployed by releasing a constraint disposed about the filter. In still other embodiments, the filter 354 is deployed by moving the filter in a direction against a flow of bodily fluid (e.g., against the flow of blood within a blood vessel) such that the fluid causes the filter to deploy.

Returning to the flow chart shown in FIG. 17, a portion of tissue T is dislodged from within the bodily lumen, 830. The tissue T can be dislodged in any manner described herein. For example, in some embodiments, the fiber 340 is moved within the bodily lumen when in its second configuration to dislodge the tissue T. The fiber 340 can be moved distally, proximally, and/or rotationally to engage and/or dislodge the tissue T from the wall of the bodily lumen.

Returning to the flow chart shown in FIG. 17, in some embodiments, the method optionally includes capturing a portion of the dislodged tissue, 835. The portion of dislodged tissue T can be captured in any manner described herein. For example, in some embodiments, a portion of the dislodged tissue T is captured within the helical coils of the fiber 340 and/or between the fiber 340 and the second elongate member 318. In another example, a portion of the dislodged tissue T can be captured by the filter 354.

Returning to the flow chart shown in FIG. 17, in some embodiments, the method optionally includes removing a portion of the dislodged tissue T from the bodily lumen, 840. The portion of dislodged tissue T can be removed in any manner described herein. For example, in some embodiments, the dislodged tissue T is aspirated from within the bodily lumen. In another example, in some embodiments, the captured tissue T is removed by withdrawing the second elongate member 318, as well as the fiber 340 and filter 354 coupled to the second elongate member, from the bodily lumen, thereby removing the captured tissue T from the body of the patient.

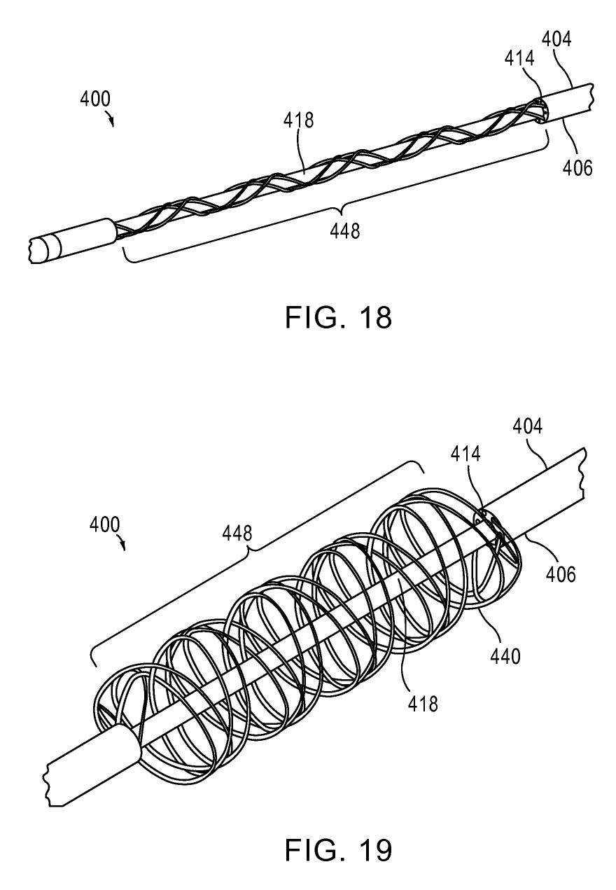

An apparatus 400 according to another embodiment is illustrated in FIGS. 18 and 19. The apparatus 400 includes a first elongate member 404, a second elongate member 418 and a set of tissue disruptors 448. The first elongate member 404 is similar in many respects to the first shaft 204, 304 described above. The first elongate member 404 includes a proximal end portion (not shown) and a distal end portion 406 and defines a first lumen (not shown), a second lumen (not shown), and a third lumen 414. The first lumen is similar in many respects to the central lumen 210, described above. The second and third lumens are similar in many respects to the pressure lumens 212, 212′, 212″, described above. The third lumen 414 of the first elongate member 404 is open at the distal end portion 406 of the first elongate member 404.

The apparatus 400 is configured to deliver a therapeutic agent, such as an anti-clotting agent, into the bodily lumen. For example, the apparatus 400 is configured to deliver the therapeutic agent via the third lumen 414 of the first elongate member 404. The therapeutic agent can be introduced into the third lumen 414 of the first elongate member 404 via a port of a valve, such as the second port 268 described above with respect to valve 260.

The set of tissue disruptors 448 are similar in many respects to the set of tissue disruptors 248 described above with respect to FIGS. 3-8, except the set of tissue disruptors 448 of apparatus 400 are in a braided configuration, rather than wound in the same direction. The set of tissue disruptors 448 is configured to be stiffened and moved from a first configuration having a first lateral dimension (shown in FIG. 18) to a second configuration having a second lateral dimension greater than the first lateral dimension (shown in FIG. 19) as described above with respect to tissue disruptors 240, 240′, 240″. The set of tissue disruptors 448 are used to dislodge an occlusive tissue T from within a bodily lumen. The set of tissue disruptors 448 are configured to capture a portion of the dislodged tissue T within the matrix and/or open space created by the braided set of tissue disruptors in the second configuration and/or between the set of tissue disruptors 448 and the second shaft 418.

The apparatus 400 can also be configured to deliver a therapeutic agent into the bodily lumen via the set of tissue disruptors 448. For example, the set of tissue disruptors 448 can be configured to release the therapeutic agent as the set of tissue disruptors 448 engages and/or dislodges the occlusive tissue T. At least one tissue disruptor 440 can define a lumen (not shown) in fluid communication with a lumen (e.g., the second lumen 412) of the first elongate member 404. The first elongate member 404 is configured to allow passage of a therapeutic agent via its second lumen 412 and into the lumen of the at least one tissue disrupter 440. The tissue disrupter 440 defines at least one small opening (not shown) through which the therapeutic agent can be conveyed. The opening can be a laser drilled hole configured to release the therapeutic agent. In other embodiments, however, the tissue disruptor 440 can be configured to elute a therapeutic agent, such as from a coating applied to an outer surface of the tissue disruptor.

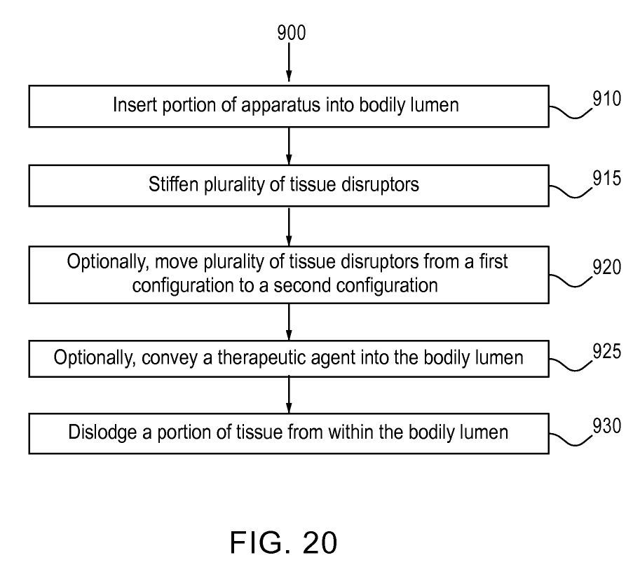

FIG. 20 is a flow chart of a method 900 of removing tissue T utilizing the apparatus 400 according to an embodiment. The method illustrated in FIG. 20 is discussed with reference to FIGS. 18 and 19. Although the method 900 is discussed with reference to the apparatus 400, the method 900 can be performed with any suitable apparatus. For example, the method 900 can be performed by any of the medical devices disclosed herein. The method includes inserting a portion of an apparatus into a bodily lumen, 910. For example, a portion of the apparatus 400 including the set of tissue disruptors 448 and the first and second elongate members 404, 418, respectively, as illustrated in FIGS. 18 and 19, can be inserted into a vessel of a patient.

Referring to the flow chart shown in FIG. 20, a set of tissue disruptors is stiffened, 915. The set of tissue disruptors 448 can be stiffened in any manner described herein. For example, the set of tissue disruptors 448 can be stiffened by conveying a fluid into lumens (not shown) of each tissue disrupter of the set of tissue disruptors. In another example, the set of tissue disruptors 448 can be stiffened by increasing the pressure of a fluid contained within a lumen of each tissue disruptor of the set of tissue disruptors.

Referring to the flow chart shown in FIG. 20, in some embodiments, the method optionally includes moving a set of tissue disruptors from a first configuration to a second configuration, 920. The set of tissue disruptors can be moved from its first configuration to its second configuration in any manner described herein. For example, in some embodiments, the stiffened set of tissue disruptors 448 is moved from its first configuration having a first lateral dimension (FIG. 18) to its second configuration having its second lateral dimension (FIG. 19). In its second configuration, the set of tissue disruptors 448 can engage the bodily tissue T to be removed and/or the wall of the bodily lumen.

Referring to the flow chart shown in FIG. 20, in some embodiments, the method optionally includes conveying a therapeutic agent into the bodily lumen, 925. For example, in some embodiments, a therapeutic agent is conveyed into the bodily lumen via the third lumen 414 of the first elongate member 404. In some embodiments, the therapeutic agent is conveyed into the bodily lumen via small openings (not shown) in at least one tissue disrupter of the set of tissue disruptors 448. In some procedures, for example, a user may convey a therapeutic agent configured to help break up thrombotic tissue T into a blood vessel of a patient.

Referring to the flow chart shown in FIG. 20, a portion of tissue T is dislodged from within the bodily lumen, 930. The tissue T can be dislodged in any manner described herein. For example, in some embodiments, the set of tissue disruptors 448 is engaged with the tissue T and manipulated to dislodge the tissue T from within the bodily lumen. A portion of the dislodged tissue T can then be removed from the bodily lumen via any of they methods described herein. In some embodiments, the set of tissue disruptors 448 is returned to its first configuration to have a narrower profile for removal of the apparatus 400 from the bodily lumen. The apparatus 400 is then removed from the body of the patient.

Although methods 700, 800, and 900 have been described with reference to apparatus 200, 300 and 400, respectively, it should be understood that any method of the present invention can be performed with any apparatus according to any embodiment.

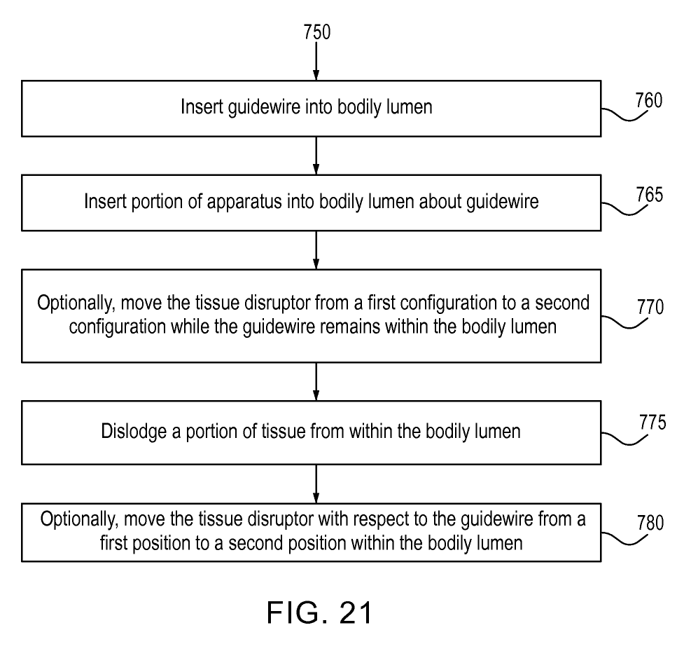

FIG. 21 is a flow chart of a method 750 of dislodging a bodily tissue within a bodily lumen according to an embodiment. The method 750 can be performed using a medical device configured to disrupt a bodily tissue of the types shown and described herein, or any suitable combination thereof. Referring to FIG. 21, a guidewire is inserted into a bodily lumen, 760. The guidewire can be, for example, inserted into a bodily lumen such that the guidewire extends through an occlusive material within the bodily lumen.

Referring to the flow chart of FIG. 21, at least a portion of the medical device is inserted into the bodily lumen about the guidewire, 765. For example, the medical device can include a shaft (e.g., shaft 204) and a tissue disruptor (e.g., tissue disruptor 240) coupled to the shaft. In some embodiments, a lumen defined by the shaft is disposed about the guidewire. Said another way, at least a portion of the guidewire is received in the lumen of the shaft. The tissue disrupter can also be at least partially disposed about and/or proximate to the guidewire. At least a portion of the shaft and/or the tissue disruptor is inserted into the bodily lumen about the guidewire.

Referring to the flow chart of FIG. 21, the method optionally includes moving the tissue disrupter from a first configuration to a second configuration while the guidewire remains within the bodily lumen, 770. In some embodiments, movement of the tissue disruptor from its first configuration to its second configuration occurs with respect to the guidewire. The tissue disrupter can be moved from any first configuration described herein to any second configuration described herein. For example, in some embodiments, the tissue disruptor is moved from a first configuration having a first lateral dimension to a second configuration having a second lateral dimension different than the first lateral dimension. In another example, in some embodiments, the tissue disrupter is moved from a first configuration having a first stiffness to a second configuration having a second stiffness different than the first stiffness.

In some embodiments, a position of the guidewire within the bodily lumen can be substantially maintained when the tissue disruptor is moved from its first configuration to its second configuration. Similarly stated, in some embodiments, the guidewire does not move relative to the bodily lumen when the tissue disruptor is moved from its first configuration to its second configuration. In other embodiments, the guidewire can move within the bodily lumen when the tissue disrupter is moved from its first configuration to its second configuration. For example, in some embodiments, the guidewire can be used to move the tissue disrupter from its first configuration to its second configuration.

Referring to the flow chart of FIG. 21, at least a portion of bodily tissue is dislodged from within the bodily lumen while the guidewire remains within the bodily lumen, 775. Said another way, at least a portion of bodily tissue is dislodged from within the bodily lumen when the medical device is disposed within the bodily lumen about the guidewire. In some embodiments, the tissue disrupter of the medical device can be configured to disrupt, break up, macerate, and/or dislodge a bodily tissue and/or an occlusive material from within the bodily lumen of the patient. For example, a user can dislodge the tissue by moving the tissue disrupter within the bodily lumen and/or relative to the occlusive material to dislodge the tissue with the tissue disrupter in its second configuration. For example, the tissue disruptor can be moved towards a wall of the bodily lumen to engage and/or dislodge the tissue from the bodily lumen. Additionally, a portion of the medical device including the tissue disrupter can be moved in at least one of a proximal direction, a distal direction, and/or a rotational direction as the tissue disrupter engages the tissue to agitate and dislodge the tissue, as described above with respect to FIG. 13. In another example, the medical device can dislodge the portion of the bodily tissue by applying a suction having a sufficient force to dislodge the portion of the bodily tissue. In yet another example, the medical device can be configured to deliver a therapeutic agent (e.g., a drug) formulated to dislodge the portion of the bodily tissue.

In some embodiments, the position of the guidewire within the bodily lumen can be maintained when the bodily tissue is dislodged from within the bodily lumen. Similarly stated, the guidewire does not move relative to the lumen when the bodily tissue is disrupted from within the bodily lumen. For example, in some embodiments, the guidewire can be used to facilitate disruption of the bodily tissue with the tissue disrupter.

Optionally, the medical device can be removed from the bodily lumen of the patient about (or over) the guidewire. For example, the guidewire can be maintained within the bodily lumen as the medical device is removed from the bodily lumen. Optionally, the tissue disrupter can be moved from its second configuration to its first configuration (e.g., prior to removing the medical device from the body) while the guidewire remains within the bodily lumen. For example, in some embodiments, the tissue disrupter can be moved from its second configuration to its first configuration for removal of the medical device from the bodily lumen.

In some embodiments, the method can optionally include moving the tissue disrupter to a second position within the bodily lumen, 780. The moving can include, for example, moving the tissue disrupter in at least one of a translational direction or a rotational direction with respect to the guidewire. For example, the tissue disrupter can be translationally moved in a first direction to advance the tissue disrupter within the bodily lumen. Moving the tissue disrupter can be done, for example, if the bodily tissue (or other occlusive material) is not fully dislodged and/or to dislodge a second occlusive material (e.g., a second thrombus) within the bodily lumen. In such embodiments, because the guidewire position is maintained, the tissue disrupter can be moved (e.g., advanced or retreated) without the need to repeat operation of inserting the guidewire.

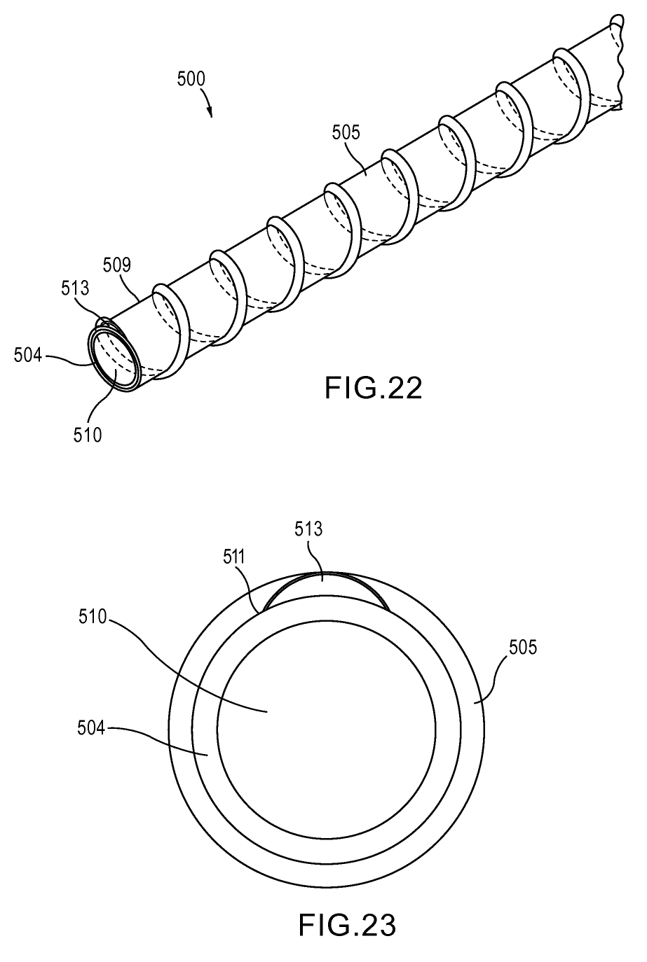

A portion of an apparatus 500 according to an embodiment is illustrated in FIGS. 22 and 23. The apparatus 500 is substantially similar to apparatus 200, 300, and 400 described above. The apparatus 500 includes a shaft 504 and a sheath 505. The shaft 504 defines a lumen 510. The sheath 505 is configured to help selectively stiffen the shaft 504. The sheath 505 includes a proximal end portion (not shown), a distal end portion 509, and defines a lumen 511. The lumen 511 of the sheath 505 is configured to receive at least a portion of the shaft 504. An interior surface of the sheath 505 and an outer surface of the shaft 504 collectively define a helically configured channel 513 extending from the proximal end portion of the sheath to the distal end portion 509 of the sheath. The channel 513 can be in fluid communication with a lumen of a tissue disrupter of the types shown and described herein, or can be sealed closed at the distal end portion 509 of the sheath 505. The channel 513 is configured to contain a fluid and has a substantially constant volume. The proximal end portion of the sheath 505 is coupled to a source of pressurized fluid. When a first pressure of the fluid contained within the channel 513 is increased, the sheath 505 is stiffened. The stiffness of the sheath 505 can be adjusted by increasing and/or decreasing the pressure of the fluid within the channel 513 using the source of pressurized fluid. In some embodiments, the sheath 505 is coupled to the outer surface of the shaft 504, for example, by bonding, ultrasonic welding, heat, glue, or any other known means for coupling.

Although the sheath 505 is illustrated and described as including a channel having a helical configuration that is substantially uniform along the length of the sheath, in other embodiments, the helical configuration of the channel can be differently configured. In some embodiments, the sheath includes a channel having a helical configuration in which the pitch of the helix varies along the length of the sheath. For example, the channel can be configured with a shorter helical pitch at the proximal end portion of the sheath, which provides more revolutions or coils of the channel along a given length, and with a longer helical pitch at the distal end portion of the sheath to provide fewer revolutions or coils of the channel along a given length. When a pressure of a fluid contained within the channel is increased, the proximal end portion of the sheath with the shorter helical pitch will be stiffer than the distal end portion of the sheath with the longer helical pitch. Such a configuration allows for variation of the stiffness along the length of the sheath and first shaft. A sheath having spatially variable stiffness can facilitate advancement of the first shaft within a bodily lumen of a patient. For example, when navigating through delicate and/or tortuous anatomy, a user can increase the pressure of the fluid within the channel so that the user can apply a greater push force to the proximal end portions of the sheath and first shaft while allowing the distal end portions of the sheath and first shaft to remain more flexible for navigating turns within the bodily lumens.

Additionally, although the sheath 505 is illustrated and described as including a helical channel, in other embodiments, the sheath can define a channel of a different pattern. For example, in some embodiments, the sheath defines a channel that is linear. In other embodiments, the sheath defines a channel that is curved, zig-zagged, or any other suitable pattern. Furthermore, an apparatus according to the invention can include more than one sheath, for example, to create a variety of channel patterns.

Although the tissue disruptors (e.g., tissue disruptors 240, 240′, 240″) have been illustrated and described herein as having a proximal end portion (e.g., proximal end portion 242, 242′, 242″) coupled to a distal end portion (e.g., distal end portion 206) of a first shaft (e.g., first shaft 204) and a distal end portion (e.g., distal end portion 244, 244′, 244″) coupled to a distal end portion 220 of a second shaft (e.g., second shaft 218), in other embodiments, an apparatus can include a tissue disrupter coupled to a medical device in any suitable orientation or fashion.

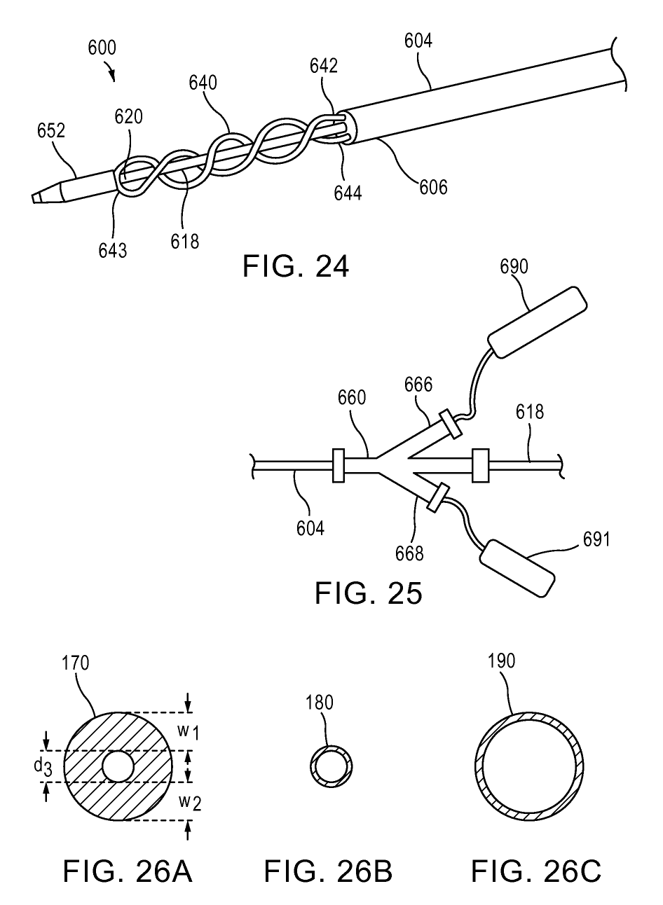

For example, as illustrated in FIGS. 24-25, an apparatus 600 according to an embodiment includes a first shaft 604, a second shaft 618, a tissue disrupter 640, a dilator 652, and a valve 660. The first shaft 604, the second shaft 618, the dilator 652, and the valve 660 are similar in many respects to the first shaft 204, second shaft 218, dilator 252, and valve 260, respectively, described above with respect to FIGS. 3-8, and are therefore not described in detail. The second shaft 618 is at least partially received in a lumen (not shown) defined by the first shaft 604. The dilator 652 is coupled to a distal end portion 620 of the second shaft 618. The second shaft 618 and dilator 652 are movable with respect to the first shaft 604. For example, the second shaft 618 and dilator 652 can be collectively movable with respect to the first shaft 604 as described above with respect to apparatus 200.

The tissue disrupter 640 includes a first end 642, a second end 644, and a central portion 643 disposed therebetween. The tissue disrupter 640 defines a lumen (not shown) extending therethrough. The tissue disrupter 640 is similar in many respects to the tissue disruptors and/or fibers described herein (e.g., tissue disruptors 240, 240′, 240″ and/or fiber 340). The tissue disrupter 640 differs from the tissue disrupter 240, however, in that each of the first end 642 and the second end 644 of the tissue disrupter 640 is coupled to a distal end 606 of the first shaft 604.

The central portion 643 of the tissue disruptor 640 is coupled to at least one of the dilator 652 or a distal end 620 of the second shaft 618. In this manner, the central portion 643 of the tissue disruptor 640 can be moved proximally and/or distally with respect to the distal end portion 606 of the first shaft 604, for example, as described above with respect to movement of the distal end portions 246, 246′, 246″ of the tissue disruptors 240, 240′, 240″ with respect to the distal end portion 206 of the first shaft 204. Similarly stated, the central portion 643 can move relative to the first end 642 and the second end 644 of the tissue disruptor 640.

The first end 642 of the tissue disrupter 640 is fluidically coupled to a first lumen (not shown) of the first shaft 604. The first lumen of the first shaft 604 can be fluidically coupled to a source of pressurized fluid 690 via a port 666 of the valve 660, as shown in FIG. 25. The second end 644 of the tissue disrupter 640 is fluidically coupled to a second lumen (not shown) of the first shaft 604. In some embodiments, the second lumen of the first shaft 604 is fluidically coupled to a reservoir 691 configured to receive a fluid from the second lumen of the first shaft 604 via the valve 660. In this manner, the apparatus 600 can define a fluid pathway from the source of pressurized fluid 690, through the tissue disruptor 640, to the reservoir 691 configured to receive the fluid. Each of port 666 and port 668 of valve 660 can be closed to prevent release of a fluid from the lumen of the tissue disruptor 640 via one of the lumens (e.g., the first lumen or the second lumen) of the first shaft 604.

The tissue disrupter 640 is configured to be selectively stiffened. The stiffness of the tissue disrupter 640 can be increased and/or decreased in any manner of stiffening described herein. For example, the tissue disruptor 640 can be selectively stiffened by increasing a pressure of a fluid disposed within the lumen of the tissue disrupter. Because the ports 666, 668 can be closed during the procedure, in some embodiments, there can be substantially no flow of fluid within the lumen of the tissue disruptor 640 when the tissue disrupter is selectively stiffened. Similarly stated, in some embodiments, the tissue disrupter 640 defines a closed system (e.g., a system that is fluidically isolated from an area outside of the tissue disrupter 640).

In some embodiments, the tissue disrupter 640 can be shipped from a manufacturing facility to an end-user (e.g., a physician) with a storage fluid disposed in the lumen of the tissue disrupter 640. The storage fluid can, for example, be configured to maintain the patency of the lumen of the tissue disruptor 640 during shipping and/or until use in a medical procedure. The storage fluid can be any suitable material and/or fluid for being disposed in the lumen of the tissue disrupter 640 during shipping, such as, for example, the types of fluid described herein with reference to the apparatus 200.

The storage fluid within the lumen of the tissue disrupter 640 is removed and/or replaced with a working fluid prior to use of the tissue disrupter 640 to dislodge bodily tissue within a body of a patient. In some procedures, for example, the storage fluid is removed from lumen of the tissue disrupter 640 prior to insertion of the tissue disrupter 640 into the body of the patient. In other procedures, the storage fluid is removed from lumen of the tissue disrupter 640 after insertion of the tissue disrupter 640 into the body of the patient (e.g., before dislodging of the bodily tissue).

The storage fluid can be removed from the lumen of the tissue disrupter 640 in any suitable manner. In some procedures, the storage fluid is removed by flushing the storage fluid through the fluid pathway within the apparatus 600. For example, the storage fluid can be flushed out of the lumen of the tissue disrupter 640 by opening the ports 666, 668 of the valve 660 and allowing the working fluid to flow from the source of pressurized fluid 690 through the fluid pathway of the apparatus to the reservoir 691 configured to receive the fluid. Such a fluid flow is configured to push, or flush, the storage fluid through the apparatus 600 and out of the valve 660 to the reservoir 691 configured to receive the fluid.

The lumen of the tissue disrupter 640 can be flushed with any suitable material. In some procedures, for example, the lumen of the tissue disrupter 640 is flushed with saline, air, any fluid described herein from a source of pressurized fluid described herein (e.g., a fluid from the source of pressurized fluid 290), or any combination thereof. After the lumen of the tissue disrupter 640 is flushed, the working fluid (e.g., a contrast fluid that is viewable with an imaging device) remains in the lumen of the tissue disrupter 640 and in the first and second lumens of the first shaft 604. The ports 666, 668 of the valve 660 are shut. In this manner, the tissue disrupter 640 defines a closed system. In other words, there is substantially no flow of the working fluid within the lumen of the tissue disrupter 640. The stiffness of the tissue disrupter 640 can be changed, as described herein.

In other procedures, the storage fluid is removed by suction. For example, in some embodiments, the tissue disrupter 640 contains ambient air. In other words, the tissue disruptor 640 can contain an amount of air without being intentionally filled with the air by the manufacturer. During some procedures, the ambient air is removed by suction, for example, prior to selectively stiffening the tissue disrupter 640. In some embodiments, an aspirator (not shown) is coupled to the port 668 of valve 660. The aspirator is configured to provide a suction to remove the storage fluid from the lumen of the tissue disrupter 640 via the second lumen of the first shaft 604. In other embodiments, the aspirator is coupled to the port 666 of valve 660. In this manner, the aspirator is configured to provide a suction to remove the storage fluid from the lumen of the tissue disruptor 640 via the first lumen of the first shaft 604. For example, in some embodiments, the valve 660 is fluidically coupled to a device (not shown) that includes the source of pressurized fluid 690 and the aspirator. As the aspirator of the device is used to remove the storage fluid, an at least partial vacuum is produced within the lumen of the tissue disruptor 640. The source of pressurized fluid 690 is then used to deliver the working fluid into the at least partial vacuum within the lumen of the tissue disrupter 640.

In some embodiments, for example, each of the first lumen and the second lumen of the first shaft 604 are fluidically coupled to the device including the source of pressurized fluid 690 and the aspirator via the port 666 of the valve 660. In this manner, the storage fluid can be removed from the lumen of the tissue disrupter 640 concurrently via each of the first lumen and the second lumen of the first shaft 604 via the aspirator of the device to produce a vacuum within the lumen of the tissue disruptor 640. Also in this manner, the working fluid can be delivered to the lumen of the tissue disrupter 640 from each of the first end 642 (via the first lumen of the first shaft 604) and the second end 644 (via the second lumen of the first shaft 604) of the tissue disruptor 640.

The first shafts (or first elongate members) as described herein (e.g., first shaft 204, first elongate member 304, first shaft 604) can be constructed from any suitable materials or combination of materials. For example, in some embodiments, the first shaft 204 can be constructed from a polymer such as, for example, polyamide, polytetrafluoroethylene (PTFE), low friction polytetrafluoroethylene (e.g., the product sold under the trademark PD Slick™), fluoroethylkene (FEP), perfluoroalkoxy (PFA), polyvinylidene fluoride (PVDF), ethylene tetrafluoroethylene (ETFE), polyether block amide (PEBA) (e.g., 25-72 D Pebax®), nylon (e.g., nylon, or a product sold under one of the trademarks Zytel®, Grilamid®, Rislan®, Vestamid®), ethylene-tetrafluoroethylene-hexafluoropropylene-fluoroterpolymer (EFEP), high density polyethylene (HDPE), low density polyethylene (LDPE), 1,1-diethylsilacylcobutane (EtSB), another thermoplastic polymer, or any combination thereof. In some embodiments, for example, the first shaft 204 includes an inner liner (not shown) defining at least a portion of the central lumen 210. The inner liner can be formed of a first material, and the other portions of the first shaft 204 can be formed of the same material, or one or more different materials. In other embodiments, the first shaft 204 can be constructed from a metal, rubber, glass, or any other suitable biocompatible material.

Similarly, the second shafts (or second elongate members) as described herein (e.g., second shaft 218, second elongate member 318, second shaft 618) can be constructed from any suitable materials or combination of materials. For example, in some embodiments, the second shaft 218 can be constructed from a polymer such as, for example, any of the polymers listed above with respect to the first shaft 204. In other embodiments, the second shaft 218 can be constructed from a metal, rubber, glass, or any other suitable biocompatible material. In other embodiments, the second shaft 218 can include a reinforcement member (not shown in FIGS. 3-8), such as for example, a braid, braided mesh, coil, additional polymeric layer, or the like, or any combination thereof. For example, the reinforcement member can include a coil extending along a portion of the second shaft and terminating at a braid. In another example, the reinforcement member can include a braid disposed over a coil. In still another example, the reinforcement member can include a coil over a braid. The reinforcement member can be constructed of any suitable material such as, for example, glass, stainless steel, nitinol, nylon, tungsten, tungsten rhenium, polymer, impregnated polymer, or the like, or any combination thereof. In some embodiments, the second shaft includes an inner shaft portion and an outer shaft portion (not shown in FIGS. 3-8). The inner shaft portion can include, for example, a liner. The outer shaft portion can be, for example, an overlaying layer. In some embodiments, the inner shaft portion is constructed of a first material, such as a material described above with respect to the first shaft 204, and the outer shaft portion is constructed of a second material, such as another of the materials described above with respect to the first shaft. In some embodiments, the marker 352 can be constructed of any suitable material such as, for example, glass, stainless steel, nitinol, impregnated nylon, tungsten, tungsten rhenium, impregnated polymer, platinum, gold, silver, titanium, iridium palladium, rhenium, or the like, or any combination thereof.

Similarly, the aspiration shaft 228 as described herein can be constructed from any suitable materials or combination of materials. For example, in some embodiments, the aspiration shaft 228 can be constructed from a polymer such as, for example, any of the polymers listed above with respect to the first shaft 204. In other embodiments, the aspiration shaft 228 can include a reinforcement member (not shown in FIGS. 3-8), such as for example, a reinforcement member similar to a reinforcement described above with respect to the second shaft 218. In some embodiments, the aspiration shaft 228 includes an inner shaft portion and an outer shaft portion (not shown in FIGS. 3-8), for example, an inner shaft portion and an outer shaft portion similar to the inner shaft portion and outer shaft portion described above with respect to the second shaft 218. In some embodiments, the aspiration shaft 228 includes a marker such as, for example, a marker band (not shown). The marker band can be constructed of any suitable material such as, for example, a material described above with reference to the second shaft 218.

The tissue disruptors (or fiber) as described herein (e.g., tissue disruptors 240, 240′, 240″, fiber 340, tissue disrupter 640) can be constructed from any suitable biocompatible materials or combination of materials. For example, in some embodiments, the tissue disruptors 240, 240′, 240″ can be constructed from a polymer such as, for example, polyether block amide (PEBA) (e.g., PEBA having a Shore Hardness of 25-72, or the product sold under the trademark Pebax®), nylon (e.g., nylon 6 or a product sold under one of the trademarks Zytel® Grilamid®, Rislan®, or Vestamid®), fluoroethylkene (FEP), polytetrafluoroethylene (PTFE), ethylene-tetrafluoroethylene-hexafluoropropylene-fluoroterpolymer (EFEP), high density polyethylene (HDPE), low density polyethylene (LDPE), 1,1-diethylsilacylcobutane (EtSB), another thermoplastic polymer, or any combination thereof. In other embodiments, the tissue disruptors 240, 240′, 240″ can be constructed from any suitable metallic and/or non-metallic material configured to engage the wall of a bodily lumen without damaging the wall. Said another way, the tissue disruptors 240, 240′, 240″ can be constructed of a material that will not strip, shave, or otherwise damage the vessel wall. For example, the tissue disruptors 240, 240′, 240″ can be constructed of a suitable flexible material.

The components of an apparatus as described herein (e.g., apparatus 200, apparatus 300, apparatus 400) can have any suitable size suitable for deployment and use within a bodily lumen as described herein. For example, in some embodiments, the first shaft 204 has a length of approximately 10 cm to 300 cm and a wall thickness of approximately 0.006 cm to 0.05 cm. In other embodiments, the first shaft 204 has a length less than 10 cm and/or a wall thickness of less than 0.008 cm. In still other embodiments, the first shaft 204 has a length greater than 300 cm and/or a wall thickness of greater than 0.008 cm. In some embodiments, for example, an inner liner (not shown) of the first shaft 204 is 0.001 cm to 0.0015 cm in thickness.

In another example, in some embodiments, the central lumen 210 of the first shaft 204 has a lateral cross-sectional dimension of 0.042 cm to 0.127 cm. In other embodiments, the central lumen has a lateral cross-sectional dimension of less than 0.042 cm. In still other embodiments, the central lumen of the first shaft 204 has a lateral cross-sectional diameter of greater than 0.127 cm. In yet another example, in some embodiments, each pressure lumen 212, 212′, 212″ of the first shaft 204 can have a circular cross-sectional shape with an internal diameter of 0.010 cm to 0.005 cm. In other embodiments, the pressure lumens 212, 212′, 212″ can each have an internal diameter less than 0.010 cm. In still other embodiments, each pressure lumen 212, 212′, 212″ can have an internal diameter of greater than 0.025 cm. In some embodiments, each pressure lumen 212, 212′, 212″ of the first shaft 204 can have a non-circular cross-sectional shape such as, for example, an oval, hexagon, octagon, or diamond. A pressure lumen 212, 212′, 212″ having a non-circular cross-section can have a perimeter size substantially equal to the perimeter of a pressure lumen 212, 212′, 212″ have any of the foregoing internal diameters.

Similarly, the second shaft (or second elongate member) 218, 318, 418 can have any suitable size suitable for deployment and use within the bodily lumen. For example, in some embodiments, the second shaft 218 can be approximately 10 cm to 300 cm in length. In other embodiments, the second shaft 218 can be less than 10 cm in length. In still other embodiments, the second shaft 218 can be greater than 300 cm in length. In another example, in some embodiments, the second shaft 218 can have a wall thickness of approximately 0.006 cm to 0.0508 cm. For example, the second shaft 218 can have an outer shaft portion that is approximately 0.006 cm to 0.0508 cm thick and an inner shaft portion that is approximately 0.006 cm to 0.008 cm thick. In other embodiments, the second shaft 218 can have a wall thickness less than 0.006 cm. In still other embodiments, the second shaft 218 can have a wall thickness greater than 0.0508 cm. In yet another example, in some embodiments, the second shaft 218 can include an outer shaft portion (e.g., an outer layer, not shown), having an length of approximately 1 to 280 cm. In some embodiments, the outer shaft portion includes a length of material from 1 to 280 cm that is adjacent or proximate to a length of a second material that is from 1 to 280 cm. In some embodiments, the marker 352 of the second elongate member 318 has a length of approximately 0.1 mm to 1 cm and a wall thickness of 0.006 cm to 0.025 cm.

Similarly, the aspiration shaft 228 can have any suitable size suitable for deployment and use within the bodily lumen. For example, in some embodiments, the aspiration shaft 228 can have a size configuration similar to those described above with respect to the second shaft 218. In another example, in some embodiments, outer shaft portion of the aspiration shaft 228 includes a first material extending along a length of the aspiration shaft of about 1 to 300 cm, and a second material extending along a length of the aspiration shaft of about 1 to 300 cm. In some embodiments, the aspiration shaft 228 has an outer diameter of 5 to 9 French (Fr), which is substantially equivalent to 1.67 mm and 3 mm, respectively. The marker band of the aspiration shaft can also be of any suitable size, such as a size described above for marker 352 with respect to the second elongate member 318.

In another example, a reinforcement member of a second shaft can have any suitable size and configuration for providing reinforcement to the second shaft. In some embodiments, for example, the reinforcement member has a length of approximately 1 to 300 cm. The reinforcement member can include a first portion, for example a coil, having a length of approximately 5 to 40 cm, and a second portion, for example a braid, having a length of approximately 40 to 275 cm. The reinforcement member can include any number of component strands, and each component strand can have a thickness of approximately 0.003 cm to 0.008 cm and a width of approximately 0.006 cm to 0.010 cm. In some embodiments, the reinforcement member is a braid or braided mesh with approximately 2 to 64 component strands. A braided reinforcement member can have a porosity of, for example, approximately 32 pores per inch (ppi) to 120 ppi. In other embodiments, for example, the reinforcement member is a coil having a component strand that has an outer circumference of approximately 0.003 cm to 0.010 cm. A coiled reinforcement member can have a pitch of approximately 0.006 cm to 0.032 cm.

In some embodiments, the central lumen 210 of the first shaft 204 can be configured to facilitate placement of the first shaft 204 within the bodily lumen and/or placement of the second shaft 218 with respect to the first shaft 204. For example, in some embodiments, the first shaft 204 can include a coating. For example, a portion of the first shaft 204 defining the central lumen 210 can include a coating. The coating can be configured to facilitate movement of the second shaft 218 within the central lumen 210 of the first shaft 204. In another example, in some embodiments, the coating is a radiopaque material disposed on a portion of the first shaft 204 defining the central lumen 210. For example, the first shaft 204 can include a strip of radiopaque material disposed along a portion of the length of the first shaft 204 defining the central lumen 210. In yet another example, in some embodiments, a portion of the first shaft 204 defining the central lumen 210 includes at least one spiral groove. The spiral groove, or rifling, of the first shaft 204 defining the central lumen 210 can, for example, help provide a frictional fit between the first shaft and the second shaft 218 to help avoid unintentional movement of the second shaft within the first shaft.

The tissue disruptors and/or fibers described herein (e.g., tissue disruptors 240, 240′, 240″, 640 and/or fiber 340) can have any suitable dimensions. For example, in some embodiments, the fiber 340 is a thread-like member having a length that is substantially greater than a cross-sectional diameter of the fiber 340. In this manner, the fiber 340 is configured to have a narrow profile when in its first configuration and to permit passage of a bodily fluid between the fiber 340 and the second elongate member 318 when in its second configuration. Also in this manner, the fiber 340 is configured to break up the occluding tissue T and/or capture the dislodged tissue T within the matrix defined by the fiber 340. Specifically, in some embodiments, the fiber 340 and/or the tissue disruptors 240, 240′, 240″, 640 can have a length of approximately 1 to 600 cm and a cross-sectional diameter of approximately 0.006 cm to 0.025 cm. In other embodiments, the fiber 340 and/or the tissue disruptors 240, 240′, 240″ can each have a length greater than 600 cm and/or a cross-sectional diameter greater than 0.025 cm. In another example, in some embodiments, the fiber 340 and/or the tissue disruptors 240, 240′, 240″, 640 can each have a wall thickness of 0.0006 cm to 0.005 cm. In other embodiments, the fiber 340 and/or the tissue disruptors 240, 240′, 240″, 640 can each have a wall thickness less than 0.0006 cm. In still other embodiments, the fiber 340 and/or the tissue disruptors 240, 240′, 240″, 640 can each have a wall thickness greater than 0.005 cm.

The total wall thickness of the fiber 340 and/or the tissue disruptors 240, 240′, 240″, 640 can be considered as the difference between an outer cross-sectional diameter (e.g., the cross-sectional diameter of the outer surface of the fiber 340 and/or the tissue disruptors 240, 240′, 240″, 640) and an internal cross-sectional diameter (e.g., the cross-sectional diameter of an inner surface of the fiber 340 and/or the tissue disruptors 240, 240′, 240″, 640 that defines the lumen). For example, in some embodiments, the fiber 340 has an outer cross-sectional diameter of 0.010 cm and an inner cross-sectional diameter of 0.008 cm, and thus a total wall thickness of 0.002 cm.

The total wall thickness of the fiber 340 and/or the tissue disruptors 240, 240′, 240″, 640 can be any suitable proportion of the outer cross-sectional diameter and/or the internal cross-sectional diameter of the fiber 340 and/or tissue disruptors 240, 240′, 240″, 640, respectively. Said another way, the total wall thickness of the fiber 340 and/or tissue disruptors 240, 240′, 240″, 640 can be proportionate to a cross-sectional diameter of the lumen of the fiber 340 and/or the tissue disruptors 240, 240′, 240″, 640 (or an internal cross-sectional diameter of the fiber 340 and/or tissue disruptors 240, 240′, 240″, 640. In some embodiments, the internal cross-sectional diameter of the lumen of the fiber 340 and/or the tissue disruptors 240, 240′, 240″, 640 is associated with a volume of the lumen of the fiber and/or the tissue disruptors. As such, the ratio of the wall thickness to the internal cross-sectional diameter of the lumen can be considered to be representative of a ratio of the wall thickness to the volume of the lumen of the fiber and/or the tissue disruptors (referred to herein as a “volume to thickness ratio”). For example, referring to FIG. 26A, in some embodiments, a fiber 170 can have a wall thickness of (w₁+w₂) and an internal cross-sectional diameter of d₃. Thus, because the internal cross-sectional diameter d₃ is associated with the volume of the lumen of the fiber 170, the fiber 170 can be characterized as having a volume to thickness ratio of d₃/(w₁+w₂).

In other embodiments, the volume to thickness ratio of the fiber 340 and/or tissue disruptor 240, 240′, 240″, 640 can be characterized as the proportion of the wall thickness of a portion of the fiber 340 and/or tissue disrupter 240, 240′, 240″, 640 (e.g., wall thickness w₁ as illustrated in FIG. 26A) to the internal cross-sectional diameter (e.g., internal cross-sectional diameter d₃ as illustrated in FIG. 26A).

In some embodiments, a fiber can have a volume to thickness ratio that is characterized as being a low volume to thickness ratio, which can be a ratio within the range of approximately 0.5 to 1, For example, in some embodiments, the tissue disrupter has an internal cross-sectional diameter of about 0.00254 cm (about 0.001 inches) and a wall thickness of about 0.00508 cm (about 0.002 inches), and thus a volume to thickness ratio of about 0.5. For example, fiber 170, illustrated in FIG. 26A can be characterized as having a low volume to thickness ratio. In other embodiments, a fiber can have a volume to thickness ratio that is characterized as being a medium volume to thickness ratio, which can be a ratio within the range of approximately 1.1 to 19.9. For example, in some embodiments, the tissue disrupter has an internal cross-sectional diameter of about 0.0635 cm (about 0.025 inches) and a wall thickness of about 0.00508 cm (about 0.002 inches), and thus a volume to thickness ratio of about 12.5. In another example, the tissue disrupter can have an internal cross-sectional diameter of about 0.07112 cm (about 0.028 inches) and a wall thickness of about 0.005715 cm (about 0.00225 inches), and thus a volume to thickness ratio of about 12.4. For example, fiber 180, illustrated in FIG. 26B, can be characterized as having a medium volume to thickness ratio, e.g., as compared to the fiber 170 of FIG. 26A. In contrast, a fiber can have a volume to thickness ratio that is characterized as being a high volume to thickness ratio, which can be a ratio within the range of approximately 20 to 100. For example, in some embodiments, the tissue disrupter has an internal cross-sectional diameter of about 0.0889 cm (about 0.035 inches) and a wall thickness of about 0.00127 cm (about 0.0005 inches), and thus a volume to thickness ratio of about 70. For example, the fiber 190, illustrated in FIG. 26C, can be characterized as having a high volume to thickness ratio, e.g., as compared to fibers 170 and 180 of FIGS. 26A and 26B, respectively. The fiber and/or tissue disruptors described herein can have any suitable volume to thickness ratio.

The volume to thickness ratio can affect the performance of the fibers 170, 180, 190, for example, during use in a procedure within a bodily vessel of a patient. For example, fiber 190, which has a high volume to thickness ratio, can have a greater range of stiffness than a fiber having a low volume to thickness ratio (e.g., fiber 170). The range of stiffness includes the possible variation in stiffness of the fiber 190, for example between the stiffness of the fiber in its first configuration and the stiffness of the fiber in its second configuration. Said another way, the range of stiffness is the degree to which stiffness of the fiber 190 can be changed during use. The range of stiffness of the fiber 190 can be attributable, for example, to the volume area within the lumen that is available for pressurization. In another example, the range of stiffness of the fiber 190 can be at least partially attributed to the flexibility of the fiber in the absence of pressurization, which results from fiber 190 having a thinner wall (e.g., than fiber 170).

In another example, a fiber having a high volume to thickness ratio (e.g., fiber 170) can be more susceptible to bursting upon an increase in pressure within the lumen than a second fiber having a lower volume to thickness ratio (e.g., fiber 170, 180) in response to an identical increase in pressure in the lumen of the second fiber. Thus, the fiber can have a volume to thickness ratio that provides both sufficient burst pressure and stiffness. In some embodiments, the sufficient burst pressure is a pressure within the range of about 30 p.s.i. to about 200 p.s.i. In some embodiments, the sufficient burst pressure is a pressure within the range of about 170 p.s.i. to about 180 p.s.i. For example, the burst pressure can be about 176 p.s.i.

The tissue disruptors and fibers described above with respect to apparatus 200, 300, and 400 (e.g., tissue disruptors 240, 240′, 240″, 640, fiber 340) can be molded into the desired configuration. For example, the tissue disrupter 240 in the helical configuration can be manufactured by placing the tissue disruptor 240 about a helical mold with the desired number and spacing of the helical coils (or turns), and then increasing the temperature of the tissue disrupter 240 to a temperature below its melt temperature and held at that temperature for a given period of time. This process is referred to as annealing, and helps the tissue disrupter 240 assume its desired configuration or shape during use. Furthermore, although the tissue disruptors and fiber have been illustrated and described as being in a helical configuration and a braided configuration, in other embodiments, the tissue disruptors (or fiber) can be in any suitable configuration. For example, in some embodiments, the tissue disrupter is linear. In other embodiments, the tissue disruptors are helically wrapped in opposite directions.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Furthermore, although methods are described above as including certain events, any events disclosed with respect to one method may be performed in a different method according to the invention. Thus, the breadth and scope should not be limited by any of the above-described embodiments. While the invention has been particularly shown and described with reference to specific embodiments thereof, it will be understood that various changes in form and details may be made.

For example, although the apparatus 200, 300, and 400 have been illustrated and described as including three (apparatus 200 and 400) tissue disruptors or one fiber (apparatus 300), in other embodiments, an apparatus can include any suitable number of tissue disruptors (or fibers). For example, in some embodiments, the apparatus includes, two, four, or more tissue disruptors.

Although the tissue disruptors 240, 240′, 240″ (or fiber 340) have been described herein as having a substantially constant volume, in other embodiments, a fiber (or tissue disruptor) has a substantially constant circumference. Similarly stated, the fiber has an outside surface area that is substantially constant whether the fiber is stiffened (e.g., pressurized) or not stiffened. For example, an apparatus can include a fiber configured to be flattened (e.g., in response to a suction being applied to the tissue disruptor) to facilitate advancement into the body of the patient. In one embodiment, for example, the fiber is constructed of a non-compliant material having walls configured to deform to flatten the tissue disruptor. The fiber is configured to return to its original (e.g., cylindrical) configuration (e.g., in response to being pressurized or otherwise stiffened) to facilitate dislodgement of the occlusive material.

Although the set of tissue disruptors 448 is illustrated and described as having a substantially uniform second lateral dimension when the set of tissue disruptors is in its second configuration, in some embodiments, the set of tissue disruptors have a spatially variable lateral dimension when the set of tissue disruptors is in its second configuration. For example, in some embodiments, an apparatus (not shown) can include a set of tissue disruptors (not shown) that includes a first tissue disrupter and a second tissue disrupter. The first tissue disrupter has a greater number of turns (or revolutions or windings) about an outer surface of a shaft of the apparatus than a second tissue disruptor, which has a fewer number of turns about the shaft than the first tissue disrupter. The first tissue disrupter can be characterized as being more tightly wound about the shaft than the second tissue disruptor. When the set of tissue disruptors is moved to its second configuration having a second lateral dimension, the second tissue disrupter is radially moved away from the exterior of the shaft a greater distance than the more tightly wound first tissue disrupter is radially moved away from the exterior of the shaft. As such, the second lateral dimension of the set of tissue disruptors in its second configuration can be spatially variable, for example, depending on which portion of the set of tissue disruptors is measured to determine the second lateral dimension. In this manner, the set of tissue disruptors having greater spatial variability can engage more and/or varied regions of the occlusive bodily tissue (e.g., a thrombus).

The first shaft (e.g., first shaft 204, 404 or first elongate member 304) can include any suitable number of pressure lumens (e.g., pressure lumens 212, 212′, 212″). For example, in some embodiments, the first shaft 204 includes one, two, four, ten, or more pressure lumens. Similarly, the first shaft 404 can include any suitable number of lumens configured to convey a therapeutic agent into the bodily lumen (e.g., similar to the third lumen 414 described above). For example, the first shaft 404 can include two, three, four, or more treatment lumens.

Although the first shaft 204 has been described above as being configured to be selectively stiffened by increasing a pressure within at least one of the pressure lumens 212, 212′, 212″, in some embodiments, the first shaft 204 can be stiffened by increasing a pressure within the central lumen 210 of the first shaft 204.

In another example, although method 700 describes stiffening the first shaft 204 by introducing a fluid into the pressure lumens 212, 212′, 212″ and/or increasing the pressure of the fluid within the second lumen, in other embodiments, the first shaft 204 can be stiffened utilizing a sheath similar to the sheath 505 described above with respect to apparatus 500. In another example, the first shaft 204 can be selectively stiffened by independently stiffening one or more of the pressure lumens 212, 212′, 212″ of the first shaft 204. In this manner, the first shaft 204 can have spatially variable stiffness.

In another example, although the tissue disrupter 240 and fiber 340 have been illustrated and described above as being coupled to the second shaft 218 and second elongate member 318, respectively, by a dilator 252 and a marker 352, respectively, in other embodiments, the tissue disrupter 240 and/or fiber 340 can be coupled to the second shaft 218 or second elongate member 318 by any suitable coupling mechanism. For example, in some embodiments, the tissue disruptors 240 (or fiber 340) can be coupled to the second shaft by an adhesive, shrink tubing, a band, or the like, or any combination of the foregoing. In some embodiments, the coupling mechanism includes an ultraviolet portion or is otherwise configured for viewing the coupling of the tissue disruptor 240 and/or fiber 340 to the second shaft 218 and/or second elongate member 318 with an imaging device. Furthermore, although the apparatus 200 is described herein as including a dilator 252, in other embodiments, a dilator is not included in an apparatus.

In yet another example, although the tissue disruptors 240, 240′, 240″ have been illustrated and described as being substantially simultaneously stiffened, in other embodiments, the tissue disruptors 240, 240′, 240″ are configured to be separately or independently stiffened. In another example, a portion of the tissue disruptors 240, 240′, 240″ can be selectively stiffened (e.g., one pressure lumen 242 of the pressure lumens 242, 242′, 242″ or a portion of at least one pressure lumen 242, 242′, 242″. In this manner, the tissue disrupter 240 can have spatially variable stiffness.

Although the tissue disruptors 240, 240′, 240″ have been illustrated and described as being stiffened by increasing a pressure in the lumen 246, 246′, 246″ that has a substantially constant volume, in other embodiments, the tissue disrupter 240, 240′, 240″ is stiffened by increasing the pressure in the lumen 246, 246′, 246″ having a variable volume.

Although the portion of each tissue disrupter 240, 240′, 240″ defining its respective lumen 246, 246′, 246″ has been illustrated and described as being low or non-compliant, in other embodiments, the portion of the tissue disrupter 240, 240′, 240″ need not be low or non-compliant. For example, in some embodiments, the portion of the tissue disrupter 240, 240′, 240″ defining the lumen 246, 246′, 246″ can be high-compliant.

Although the compliance of the tissue disrupter 240 has been described above as relating to a change in pressure within the lumen 246 of the tissue disruptor, in other embodiments, the compliance of the tissue disrupter can be characterized differently. for example, in some embodiments, the compliance of a tissue disrupter can be used to characterize the change in the length of the tissue disrupter as a function of the lumen pressure. The change in length can also be referred to as the elongation percentage of the tissue disruptor. In other embodiments, the compliance of a tissue disrupter can be used to characterize the change in the diameter of the tissue disrupter as a function of the pressure within the lumen.

Although various embodiments have been described as having particular features and/or combinations of components, other embodiments are possible having a combination of any features and/or components from any of embodiments as discussed above. For example, one such embodiment includes an elongate assembly, a filter, and tissue disruptors configured to convey a therapeutic agent into a bodily lumen. In another example, an embodiment includes a braided set of tissue disruptors having distal end portions coupled to a guidewire (or a second shaft).

What is claimed is:

1. An apparatus, comprising:
an elongate member configured to be at least partially disposed within a bodily lumen; and
a tissue disrupter coupled to a distal end portion of the elongate member, the tissue disrupter configured to dislodge a tissue from within the bodily lumen, the tissue disrupter configured to be selectively stiffened.

2. The apparatus of claim 1, wherein the tissue disrupter is configured to be selectively stiffened by increasing a pressure of a fluid within a lumen defined by the tissue disrupter.

3. The apparatus of claim 1, wherein the tissue disrupter defines a lumen configured to contain a fluid, the lumen having a substantially constant volume, the fluid within the lumen having a first pressure when the tissue disrupter has a first stiffness, the fluid within the lumen having a second pressure different from the first pressure when the tissue disrupter has a second stiffness.

4. The apparatus of claim 1, wherein the elongate member is a first elongate member defining a lumen, the apparatus further comprising:
a second elongate member movably disposed within the lumen of the first elongate member, the tissue disrupter configured to be selectively stiffened when the second elongate member is moved with respect to the first elongate member.

5. The apparatus of claim 1, wherein the elongate member is a first elongate member, the apparatus further comprising:
a second elongate member at least partially disposed within a lumen of the first elongate member, a portion of the tissue disrupter disposed about an exterior surface of the second elongate member.

6. The apparatus of claim 1, wherein the tissue disrupter is one of a plurality of tissue disruptors, the plurality of tissue disruptors collectively having a first lateral dimension when the plurality of tissue disruptors is in a first configuration, the plurality of tissue disruptors collectively having a second lateral dimension greater than the first lateral dimension when the plurality of tissue disruptors is in a second configuration, the plurality of tissue disruptors configured to allow passage of a bodily fluid between the plurality of tissue disruptors when the plurality of tissue disruptors is in its second configuration.

7. The apparatus of claim 1, further comprising:
an aspiration shaft defining a lumen, a portion of the elongate member movably disposed within the lumen of the aspiration shaft, an inner surface of the aspiration shaft and an outer surface of the elongate member defining a passageway configured to receive a portion of the tissue.

8. The apparatus of claim 1, wherein the elongate member defines a center line, the tissue disrupter having a first stiffness when in a first configuration and a second stiffness when in a second configuration, the second stiffness different than the first stiffness, a portion of the tissue disrupter configured to move in a direction substantially normal to the center line when the tissue disrupter is moved from its first configuration to its second configuration.

9. The apparatus of claim 1, wherein at least a portion of the elongate member is configured to be selectively stiffened.

10. The apparatus of claim 1, wherein the tissue disrupter is configured to deliver a therapeutic agent into the bodily vessel.

11. The apparatus of claim 1, wherein the tissue disrupter is constructed from a non-metallic material.

12. The apparatus of claim 1, wherein the tissue disrupter includes a fiber defining a lumen, the fiber coupled to a distal end portion of the elongate member such that the lumen of the fiber is fluidically coupled to a lumen defined by the elongate member.

13. The apparatus of claim 1, wherein the tissue disrupter has a first stiffness when in a first configuration and a second stiffness when in a second configuration, the second stiffness different than the first stiffness, the tissue disrupter configured to contact an inner surface of a vessel defining the bodily lumen.

14. The apparatus of claim 1, wherein the tissue disruptor has a length of approximately 1 cm to 600 cm and an internal diameter of approximately 0.006 cm to 0.025 cm.

15. The apparatus of claim 1, wherein the elongate member is a first elongate member, wherein the tissue disruptor is a first tissue disruptor, the apparatus further comprising:
a second elongate member, at least a portion of the second elongate member disposed within the lumen of the first elongate member; and
a second tissue disruptor coupled to the distal end portion of the first elongate member, the tissue disruptor configured to dislodge the tissue from within the bodily lumen,
the first tissue disruptor wrapped about an outer surface of the second elongate member, the first tissue disruptor having a first number of turns about the outer surface of the second elongate member, the second tissue disruptor wrapped about the outer surface of the second elongate member, the second tissue disruptor having a second number of turns about the outer surface of the second elongate member, the second number of turns greater than the first number of turns.

16. The apparatus of claim 1, wherein the elongate member is a first elongate member, wherein the tissue disruptor is a first tissue disruptor, the apparatus further comprising:
a second elongate member, at least a portion of the second elongate member disposed within the lumen of the first elongate member; and
a second tissue disruptor coupled to the distal end portion of the first elongate member, the tissue disruptor configured to dislodge the tissue from within the bodily lumen,
each of the first tissue disruptor and the second tissue disruptor having a first configuration and a second configuration, the first tissue disruptor having a lateral dimension when the first tissue disruptor is in its second configuration, the second tissue disruptor having a lateral dimension greater than the lateral dimension of the first tissue disruptor when the second tissue disruptor is in its second configuration.

17. An apparatus, comprising:
an elongate assembly configured to be disposed within a bodily lumen, the elongate assembly including a first shaft and a second shaft, the first shaft defining a first lumen and a second lumen, a portion of the second shaft being movably disposed within the first lumen of the first shaft; and
a fiber having a proximal end portion and a distal end portion and defining a lumen, the proximal end portion of the fiber coupled to a distal end portion of the first shaft such that the lumen of the fiber is in fluid communication with the second lumen of the first shaft, the distal end portion of the fiber coupled to a distal end portion of the second shaft.

18. The apparatus of claim 17, wherein the fiber is configured to be selectively stiffened.

19. The apparatus of claim 17, wherein the lumen of the fiber is configured to contain a fluid, the lumen of the fiber having a substantially constant volume, the fluid within the lumen of the fiber having a first pressure when the fiber is in a first configuration, the fluid within the lumen of the fiber having a second pressure different than the first pressure when the fiber is in a second configuration.

20. The apparatus of claim 17, wherein the second shaft is configured to move within the first lumen of the first shaft to move a portion of the fiber in a radial direction relative to at least one of the first shaft and the second shaft.

21. The apparatus of claim 17, wherein the elongate assembly includes a third shaft, a portion of the first shaft disposed within a lumen defined by the third shaft, an inner surface of the third shaft and an outer surface of the first shaft collectively defining a suction passageway configured to receive a bodily material.

22. The apparatus of claim 17, further comprising:
a valve coupled to a proximal end portion of the elongate assembly, the valve configured to selectively control a fluid pressure within the second lumen of the first shaft.

23. The apparatus of claim 17, wherein the fiber is constructed from a substantially non-compliant material.

24. The apparatus of claim 17, wherein the fiber is constructed from a non-metallic material.

25. The apparatus of claim 17, wherein the fiber has a first stiffness when in a first configuration and a second stiffness when in a second configuration, the second stiffness different than the first stiffness, the fiber is configured to contact a wall of the bodily lumen when the fiber is in the second configuration.

26. The apparatus of claim 17, wherein:
the distal end portion of the second shaft includes a dilator; and
the fiber is a first fiber from a plurality of fibers, a distal end portion of each fiber from the plurality of fibers being coupled to the dilator.

27. A method, comprising:
inserting a distal end portion of an elongate assembly into a bodily lumen, the elongate assembly including a tissue disrupter;
increasing a stiffness of the tissue disrupter; and
dislodging a portion of a tissue from within the bodily lumen by engaging the tissue disrupter with the tissue.

28. The method of claim 27, wherein the increasing includes conveying a fluid into a lumen defined by the tissue disruptor.

29. The method of claim 27, wherein the increasing includes selectively increasing a pressure of a fluid within a lumen defined by the tissue disruptor.

30. The method of claim 27, wherein the increasing includes selectively increasing a pressure of a fluid within a lumen defined by the tissue disrupter while maintaining a volume of the tissue disrupter at a substantially constant value.

31. The method of claim 27, wherein:
the elongate assembly includes an elongate shaft; and
the increasing includes rotating a distal end portion of the tissue disrupter with respect to the elongate shaft.

32. The method of claim 27, further comprising:
inflating an expandable member coupled to the distal end portion of the elongate assembly within the bodily lumen to substantially prevent flow of a bodily fluid therethrough.

33. The method of claim 27, wherein the elongate assembly includes an elongate shaft, further comprising:
moving a distal end portion of the tissue disruptor in a proximal direction with respect to the elongate shaft, a portion of the tissue disruptor moving in a radial direction towards a wall of the bodily lumen when the distal end portion of the tissue disrupter moves in the proximal direction with respect to the elongate shaft such that the portion of the tissue disruptor contacts the tissue within the bodily lumen.

34. The method of claim 27, wherein the dislodging includes moving a portion of the elongate assembly in a first direction within the bodily lumen and a second direction within the bodily lumen different than the first direction.

35. The method of claim 27, further comprising:
capturing a portion of the tissue with the tissue disruptor; and
withdrawing the tissue disruptor and captured tissue from the bodily lumen.

36. The method of claim 27, further comprising:
aspirating, after the dislodging, the portion of the tissue through a passageway defined by an outer surface of an elongate shaft of the elongate assembly and an inner surface of an aspiration shaft disposed about a portion of the elongate shaft.

37. The method of claim 27, further comprising:
conveying a therapeutic agent into the bodily lumen via the tissue disrupter.

38. A method, comprising:
inserting a portion of a medical device into a bodily lumen about a guidewire disposed within the bodily lumen; and
dislodging at least a portion of a bodily tissue from within the bodily lumen when the guidewire remains in the bodily lumen.

39. The method of claim 38, further comprising:
moving a tissue disruptor of the medical device with respect to the guidewire from a first position within the bodily lumen to a second position within the bodily lumen, the moving be in at least one of a translation direction or a rotational direction.

40. The method of claim 38, further comprising:
moving a tissue disrupter of the medical device from a first configuration to a second configuration while the guidewire remains within the bodily lumen, the tissue disrupter configured to dislodge the portion of the bodily tissue.
